SEQUENCE LISTING (27 sequences)

SEQ ID NO: 1 - protein, 495 aa, synthetic construct
MEFGLSWLFLVAILKGVQCEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSSGGGGSGGGGSGGGGSSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 19 aa, synthetic construct
MEFGLSWLFLVAILKGVQC

SEQ ID NO: 3 - protein, 120 aa, synthetic construct [sequence omitted]

SEQ ID NO: 4 - protein, 17 aa, synthetic construct
SGGGGSGGGGSGGGGSS

SEQ ID NO: 5 - protein, 107 aa, synthetic construct [sequence omitted]

SEQ ID NO: 6 - protein, 15 aa, synthetic construct
EPKSCDKTHTCPPCP

SEQ ID NO: 7 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 8 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 741 aa, synthetic construct
MATGSRTSLLLAFGLLCLPWLQEGSAEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSGVKQTLNFDLLKLAGDVESNPGPMATGSRTSLLLAFGLLCLPWLQEGSADIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 26 aa, synthetic construct
MATGSRTSLLLAFGLLCLPWLQEGSA

SEQ ID NO: 11 - protein, 119 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVS

SEQ ID NO: 12 - protein, 331 aa, synthetic construct
SASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 22 aa, synthetic construct
VKQTLNFDLLKLAGDVESNPGP

SEQ ID NO: 14 - protein, 26 aa, synthetic construct
MATGSRTSLLLAFGLLCLPWLQEGSA

SEQ ID NO: 15 - protein, 107 aa, synthetic construct [sequence omitted]

SEQ ID NO: 16 - protein, 107 aa, synthetic construct [sequence omitted]

SEQ ID NO: 17 - protein, 26 aa, synthetic construct
MATGSRTSLLLAFGLLCLPWLQEGSA

SEQ ID NO: 18 - protein, 115 aa, synthetic construct
EVQLQQSGPELVKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTLTVSS

SEQ ID NO: 19 - protein, 331 aa, synthetic construct; identical to SEQ ID NO: 12

SEQ ID NO: 20 - skipped

SEQ ID NO: 21 - protein, 18 aa, synthetic construct
EGRGSLLTCGDVEENPGP

SEQ ID NO: 22 - protein, 26 aa, synthetic construct
MATGSRTSLLLAFGLLCLPWLQEGSA

SEQ ID NO: 23 - protein, 108 aa, synthetic construct
DIVMTQSHKFMSTSVGDRVSIICKASQDVGTAVDWYQQKPGQSPKLLIYWASTRHTGVPDRFTGSGSGTDFTLAITNVQSEDLADYFCQQYNSYPLTFGAGTKLEIKR

SEQ ID NO: 24 - protein, 106 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 733 aa, synthetic construct
MATGSRTSLLLAFGLLCLPWLQEGSAEVQLQQSGPELVKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTLTVSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSGEGRGSLLTCGDVEENPGPMATGSRTSLLLAFGLLCLPWLQEGSADIVMTQSHKFMSTSVGDRVSIICKASQDVGTAVDWYQQKPGQSPKLLIYWASTRHTGVPDRFTGSGSGTDFTLAITNVQSEDLADYFCQQYNSYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - DNA, 5333 bp, synthetic construct
  source 1..5333: mol_type = other DNA
ctggatccagatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatggtctccctatagtgagtcgtattagatcccgggggatccagtttggttaattaaaccggtgagtttcatggttacttgcctgagaagattaaaaaaagtaatgctaccttatgagggagagtcccagggaccaagatagcaactgtcatagcaaccgtcacactgctttggtcaaggagaagaccctttggggaactgaaaacagaaccttgagcacatctgttgctttcgctcccatcctcctccaacagggctgggtggagcactccacaccctttcaccggtcgtacggctcagccagagtaaaaatcacacccatgacctggccactgagggcttgatcaattcactttgaatttggcattaaataccattaaggtatattaactgattttaaaataagatatattcgtgaccatgtttttaactttcaaaaatgtagctgccagtgtgtgattttatttcagttgtacaaaatatctaaacctatagcaatgtgattaataaaaacttaaacatattttccagtaccttaattctgtgataggaaaattttaatctgagtattttaatttcataatctctaaaatagtttaatgatttgtcattgtgttgctgtcgtttaccccagctgatctcaaaagtgatatttaaggagattattttggtctgcaacaacttgatagggctcagcctctcccacccaacgggtggaatcccccagagggggatttccaagaggccacctggcagttgctgagggtcagaagtgaagctagccacttcctcttaggcaggtggccaagattacagttgacccgtacgtgcagctgtgcccagcctgccccatcccctgctcatttgcatgttcccagagcacaacctcctgccctgaagccttattaataggctggtcacactttgtgcaggagtcagactcagtcaggacacagctctagagtcgacgaattccaccatggagttcggacttagctggctgttcctggtggctattctcaaaggcgtccaatgcgaagttcagctggttgaatctggtggtggtctggttcagccgggtggttctctgcgtctgtcttgcgctgcttctggtttcaacatcaaagacacctacatccactgggttcgtcaggctccgggtaaaggtctggaatgggttgctcgtatctacccgaccaacggttacacccgttacgctgactctgttaaaggtcgtttcaccatctctgctgacacctctaaaaacaccgcttacctgcagatgaactctctgcgtgctgaagacaccgctgtttactactgctctcgttggggtggtgacggtttctacgctatggactactggggtcagggtaccctggttaccgtttctagttctggcggcggtgggagcggtggtggcggctcaggcggcggcgggtctagtgacatccagatgacccagtctccgtcttctctgtctgcttctgttggtgaccgtgttaccatcacctgccgtgcttctcaggacgttaacaccgctgttgcttggtaccagcagaaaccgggtaaagctccgaaactgctgatctactctgcttctttcctgtactctggtgttccgtctcgtttctctggttctcgttctggtaccgacttcaccctgaccatctcttctctgcagccggaagacttcgctacctactactgccagcagcactacaccaccccgccgaccttcggtcagggtaccaaagttgaaatcaaagaacctaagtcttgcgataaaacacacacatgcccaccatgtccagccccggaactcctcggcggaccgtccgtgtttctcttcccaccaaagccgaaggataccctcatgatttccaggaccccggaagtgacctgcgtggtcgtagatgtctctcacgaagaccctgaggtgaaatttaattggtatgtcgatggtgttgaagtgcataacgctaaaaccaaaccaagggaagagcaatataactctacctacagggttgtatcagtgttaactgtgcttcatcaggattggctcaacggtaaggaatataagtgcaaagtcagtaacaaggctctccctgctccaattgagaaaacaatatccaaggcaaaaggtcaaccacgggaaccacaagtctacacattgccacccagccgcgacgaacttacaaagaatcaagtttcacttacatgtctggttaaaggattctaccctagcgatatagccgtagagtgggagtcaaatgggcaacctgaaaataactataaaactacaccgccggtccttgattcagatgggtctttctttctctatagcaagctcactgtcgacaaatcccggtggcaacaagggaatgtgttttcctgtagcgtaatgcacgaagcacttcataatcactatacgcaaaagagtttgtccttatctcccgggaaatgagcggccgcctcgagagatcttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcataccggtcaagctagcgatatcaattaaccctcactaaagggagaccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatctggtaccatttaaatctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcctttttgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttatt

SEQ ID NO: 27 - DNA, 5321 bp, synthetic construct
  source 1..5321: mol_type = other DNA
ctggatccagatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatggtctccctatagtgagtcgtattagatcccgggggatccagtttggttaattaaaccggtgagtttcatggttacttgcctgagaagattaaaaaaagtaatgctaccttatgagggagagtcccagggaccaagatagcaactgtcatagcaaccgtcacactgctttggtcaaggagaagaccctttggggaactgaaaacagaaccttgagcacatctgttgctttcgctcccatcctcctccaacagggctgggtggagcactccacaccctttcaccggtcgtacggctcagccagagtaaaaatcacacccatgacctggccactgagggcttgatcaattcactttgaatttggcattaaataccattaaggtatattaactgattttaaaataagatatattcgtgaccatgtttttaactttcaaaaatgtagctgccagtgtgtgattttatttcagttgtacaaaatatctaaacctatagcaatgtgattaataaaaacttaaacatattttccagtaccttaattctgtgataggaaaattttaatctgagtattttaatttcataatctctaaaatagtttaatgatttgtcattgtgttgctgtcgtttaccccagctgatctcaaaagtgatatttaaggagattattttggtctgcaacaacttgatagggctcagcctctcccacccaacgggtggaatcccccagagggggatttccaagaggccacctggcagttgctgagggtcagaagtgaagctagccacttcctcttaggcaggtggccaagattacagttgacccgtacgtgcagctgtgcccagcctgccccatcccctgctcatttgcatgttcccagagcacaacctcctgccctgaagccttattaataggctggtcacactttgtgcaggagtcagactcagtcaggacacagctctagagtcgacgaattccaccatggagttcggacttagctggctgttcctggtggctattctcaaaggcgtccaatgcgaggtccagctgcagcagagtggtcccgagctggtaaagcctggaacgagtgtgcgcatatcatgtaaaacatccggatatacattcacagaatataccatccactgggtgaagcaatcccacgggaagtccctggaatggatcggtaatatcaacccaaacaatgggggcaccacatacaatcaaaaattcgaggacaaggcaaccctgactgtggacaagtccagctccaccgcctacatggaattgcggagcctcacctccgaggattccgctgtatattactgtgccgccggctggaattttgactattggggtcaaggaacaaccttgaccgtgtcctctagttctggcggcggtgggagcggtggtggcggctcaggcggcggcgggtctagtgatatcgtgatgacacagagccataagtttatgagtacgtcagtgggagacagagtgagcataatctgtaaagccagccaggatgtgggaactgcggtcgactggtaccagcaaaaaccaggacagtcacccaagcttttgatatactgggcgagcactaggcatacaggggtgcctgaccggtttaccggatctggatcagggacagacttcacccttgctataactaatgttcaatccgaggatcttgcagattatttctgccaacagtacaacagctaccctctgaccttcggggccggaaccaagttagagatcaaagaacctaagtcttgcgataaaacacacacatgcccaccatgtccagccccggaactcctcggcggaccgtccgtgtttctcttcccaccaaagccgaaggataccctcatgatttccaggaccccggaagtgacctgcgtggtcgtagatgtctctcacgaagaccctgaggtgaaatttaattggtatgtcgatggtgttgaagtgcataacgctaaaaccaaaccaagggaagagcaatataactctacctacagggttgtatcagtgttaactgtgcttcatcaggattggctcaacggtaaggaatataagtgcaaagtcagtaacaaggctctccctgctccaattgagaaaacaatatccaaggcaaaaggtcaaccacgggaaccacaagtctacacattgccacccagccgcgacgaacttacaaagaatcaagtttcacttacatgtctggttaaaggattctaccctagcgatatagccgtagagtgggagtcaaatgggcaacctgaaaataactataaaactacaccgccggtccttgattcagatgggtctttctttctctatagcaagctcactgtcgacaaatcccggtggcaacaagggaatgtgttttcctgtagcgtaatgcacgaagcacttcataatcactatacgcaaaagagtttgtccttatctcccgggaaatgagcggccgcctcgagagatcttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcataccggtcaagctagcgatatcaattaaccctcactaaagggagaccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatctggtaccatttaaatctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcctttttgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttatt

(19) United States
(12) Patent Application Publication
(10) Pub. No.: US 2025/0152714 A1
(43) Pub. Date: May 15, 2025

(54) MIGRATORY MODIFIED DIFFERENTIATED B CELLS FOR CANCER THERAPY

(71) Applicant: Immusoft Corporation, Seattle, WA (US)

(72) Inventors: Andres G. GRANDEA, III, Everett, WA (US); Sean W. AINSWORTH, Orchard Lake, MI (US); Kole D. MEEKER, Tulalip, WA (US); Robert S. SIKORSKI, Woodside, CA (US)

(21) Appl. No.: 18/949,362

(22) Filed: Nov. 15, 2024

Related U.S. Application Data
(60) Provisional application No. 63/599,102, filed on Nov. 15, 2023.

(51) Int. Cl. A61K 39/00; A61P 35/00; C07K 16/30; C07K 16/32; C12N 5/0781

(52) U.S. Cl. CPC A61K 40/4205; A61K 40/13; A61K 40/4276; A61P 35/00; C07K 16/3069; C07K 16/32; C12N 5/0635; C07K 2317/24; C07K 2317/622

(57) ABSTRACT

The present disclosure provides genetically modified autologous and/or allogenic B cell compositions and methods for treatment of cancer and/or metastatic cancer. B cells are modified ex vivo. The genetically modified B cell express at least one therapeutic protein, wherein the modified B cell is CD38+, CD138+, CD78+, IL-6R+, and CD27++ and wherein the modified B cell is capable of homing to bone marrow for improved efficacy of cancer treatment. In typical embodiments, the therapeutic protein is capable of binding a tumor associated antigen (TAA) located within the bone-localized cancer. The administered modified B cell composition can express and release the therapeutic protein at the cancer site, typically within bone.

### CROSS-REFERNECE TO RELATED APPLICATIONS

The present application claims priority to U.S. Provisional Application 63/599,102, filed Nov. 15, 2023; the content of which is incorporated herein by reference in its entirety.

### REFERENCE TO SEQUENCE LISTING

The contents of the electronic sequence listing (IMCO_012_001US_SeqList_ST26.xml; Size: 38,151 bytes; and Date of Creation: Nov. 14, 2024) are herein incorporated by reference in its entirety.

### FIELD OF THE INVENTION

Cancer metastasis is the primary cause of post-operation or post-therapy recurrence in cancer patients. Despite intensive efforts to develop treatments, cancer metastasis remains substantially refractory to therapy. For example, bone is one of the most common sites of metastasis of various types of human cancers (e.g., breast, lung, prostate and thyroid cancers). The occurrence of bone metastases causes serious morbidity due to intractable pain, high susceptibility to fracture, nerve compression and hypercalcemia.

B cell therapy represents an underexplored and promising cancer immunotherapy. For example, plasmablasts are of therapeutic interest because they secrete more antibodies than B cells, but less than plasma cells. Plasmablasts divide rapidly, and they continue to internalize antigens and present antigens to T cells. Plasmablasts have the capacity to migrate to sites of chemokine production (e.g., in bone marrow) whereby they may differentiate into long-lived plasma cells. Ultimately, a plasmablast may either remain as a plasmablast for several days and then die or irrevocably differentiate into a mature, fully differentiated plasma cell. Specifically, plasmablasts that are able home to tissues containing plasma cell survival niches (e.g., in bone marrow) are able to displace resident plasma cells in order to become long lived plasma cells, which may continue to secrete high levels of proteins for years.

Antibody-based immunotherapies, such as monoclonal antibodies, antibody-fusion proteins, and antibody drug conjugates (ADCs) are used to treat a wide variety of diseases, including many types of cancer. Such therapies may depend on recognition of cell surface molecules that are differentially expressed on cells for which elimination is desired (e.g., target cells such as cancer cells) relative to normal cells (e.g., non-cancer cells). Binding of an antibody-based immunotherapy to a cancer cell can lead to cancer cell death via various mechanisms, e.g., antibody-dependent cell-mediated cytotoxicity (ADCC), complement-dependent cytotoxicity (CDC), or direct cytotoxic activity of the payload from an antibody-drug conjugate (ADC).

Current cell therapy methods are promising strategies for the treatment of various diseases and disorders; however, there still remains a need in the art for the long-term treatment for many chronic diseases such as metastatic cancer. The present disclosure provides modified B cell compositions, e.g., plasmablasts, plasma cells, or memory B cells, for long term in vivo expression of therapeutic agents, e.g., therapeutic antibodies or antibody fragments, and methods for producing the B cell compositions and use in prophylactic and therapeutic applications.

### SUMMARY OF THE INVENTION

The present disclosure provides genetically modified autologous and/or allogenic differentiated B cell compositions and methods for treatment of cancer and/or metastatic cancer. Differentiated B cells are modified ex vivo using, for example, transposon systems (e.g., Sleeping Beauty). The genetically modified differentiated B cell express at least one scFv, wherein the modified differentiated B cell is CD38+, CD138+, and CD20⁻, wherein CD20⁻ refers to low or no CD20 expression, and wherein the modified differentiated B cell is capable of homing to bone marrow. In typical embodiments, the scFv can bind a tumor associated antigen (TAA). In certain embodiments, the gene that expresses the exogenous scFv is genomically incorporated. Methods for producing a population of modified differentiated B cells are described herein. The treatment of a disease in a human subject comprises administering to the human subject a composition of modified differentiated B cells. In some embodiments, the modified differentiated B cells are engrafted within the bone marrow. The administered modified differentiated B cell composition can express and release the therapeutic protein at the cancer site, typically within bone for improved therapeutic efficacy by targeting the bone directly with the differentiated B cell composition.

### BRIEF DESCRIPTION OF THE DRAWINGS

FIG. 1 shows the IDUA activity in tissue from mice sacrificed at 56 days post-treatment. There were four treatment groups of mice: (1) mice treated with only 3E6 CD4+ T cells, (2) mice treated with only 1E7 differentiated B cells IV, (3) mice treated with 1E7 PSMA-CHO cells differentiated B cells IV and 3E6 CD4+ T cells one week prior IP, and (4) mice treated with 1E7 differentiated B cells IP and 3E6 CD4+ T cells one week prior IP. Extracts were prepared from the tissues indicated in the key to the left and assayed for IDUA activity. The mean +/− S.D. is shown for N=4 in all four groups.

FIG. 2 shows the IDUA activity in tissue from mice sacrificed at 65 days post-treatment for groups dosed at 3E6 and 1E7 differentiated B cells or at 85 days for the group dosed at 3E7 differentiated B cells. Control groups were split between the two harvest time points. There were five treatment groups of mice: (1) mice treated with 3E6 differentiated B cells and 3E6 CD4+ T cells one week prior, (2) mice treated with 1E7 differentiated B cells and 3E6 CD4+ T cells one week prior, (3) mice treated with 3E7 differentiated B cells and 3E6 CD4+ T cells one week prior, (4) mice treated with only 3E6 CD4+ T cells, (5) mice not treated with either differentiated B cells or T cells. All differentiated B treatments were done via IV. Extracts were prepared from the tissues. The mean +/− S.D. is shown for N=5 in all three differentiated B cell treated groups. N=4 for all control groups.

FIG. 3 shows the IDUA activity in tissue from mice sacrificed at 3 (group 1), 6 (group II), and 6.5 months (group III) (3 m, 6 m, and 6.5 m) after the second of two injections of 2E7 differentiated B cells given 36 days after the first injection. Control groups (4 and 5) were split between the three harvest time points. All differentiated B cell treated mice received CD4+ T cells IP 7 days prior to the first differentiated B cell injection. Extracts were prepared from the tissues indicated on the horizontal axis and assayed for IDUA activity. The mean +/− S.D. is shown for N=5 for the 3 and 6 month timepoints, N=3 for the 6.5 month timepoint. N=1,1, and 2 for IDUA+/− untreated controls at 3, 6 and 6.5 months. N=1, 1, and 0 for MPSI untreated controls at 3, 6 and 6.5 months, respectively.

FIG. 4A-FIG. 4I show the IDUA activity in tissue from mice sacrificed at 2 months. There were six treatment groups: (1) 5E5 differentiated B cells, (2) 1E6 differentiated B cells, (3) 2E6 differentiated B cells, (4) 1E7 differentiated B cells, (5) MPSI untreated mice that only got T cells, and (6) vehicle treated mice (IDUA +/−). Extracts were prepared from brain (FIG. 4A), lung (FIG. 4B), liver (FIG. 4C), heart (FIG. 4D), kidney (FIG. 4E), spleen (FIG. 4F), muscle (FIG. 4G), intestine (FIG. 4H), and bone marrow (FIG. 41) and assayed for IDUA activity. All differentiated B cell treated mice received CD4+ T cells IP 7 days prior to the first differentiated B cell injection. The mean +/− S.D. is shown for N=6 in all groups except the IDUA+/− group which had an N=7.

FIG. 5 shows activated B cells expressing GFP. B cells were isolated from human blood, transduced using a measles-virus pseudotyped lentivirus encoding GFP under control of the EEK promoter, and exposed to the Baltimore culture system for 12 days. In the background, out of the plane of focus, can be observed the CD40 ligand expressing feeder cell layer. The GFP positive cells are differentiating into plasma cells and subsequently display a much larger morphology in comparison to the B cells initially isolated from whole blood.

FIG. 6 shows production of the HIV neutralizing antibody b12. Media from both the transduced and non-transduced cell cultures were tested for the presence of b12 using a Luminex detection device

FIG. 7 illustrates Herceptin antibodies expressed from a Sleeping Beauty (SB) construct comprising a EEK promoter. The full-length Herceptin antibody comprised a F2A slipping sequence between the heavy and light chain domains.

FIG. 8 illustrates Her-2 cell surface binding (Her-2 positive CHO cells) of Herceptin scFv and surface PSMA binding of J591 scFv (PSMA-CHO) as measured by flow cytometry. Herceptin scFv and J591 scFv supernatant from Sleeping Beauty (SB) transduced differentiated B cells were tested.

FIG. 9 illustrates Herceptin scFv and Herceptin H-F2A-L (full-length antibody) Her-2 binding as measured by flow cytometry. Supernatants from transiently transfected serum-free (SF) CHO cells were included as a positive expression control.

FIG. 10 shows the binding of the supernatant containing the anti-PSMA J591 scFv (left) or GFP (right) from differentiated B cells to PSMA-CHO cells.

### DETAILED DESCRIPTION OF THE INVENTION

### B Cell Compositions

The B cells used in the methods described herein include pan B cells, memory B cells, differentiated B cells, plasmablasts, and/or plasma cells. In one embodiment, the modified B cells are memory B cells. In one embodiment, the modified B cells are differentiated B cells. In one embodiment, the modified B cells are plasmablasts. In one embodiment, the modified B cells are plasma cells. In some embodiments, the B cells are a mixture of pan B cells, memory, differentiated B cells, plasmablasts, and/or plasma cells. Terminally differentiated plasma cells do not express common pan-B cell markers, such as CD19 and CD20, and express relatively few surface antigens. Plasma cells express CD38, CD78, CD138 and interleukin-6 receptor (IL-6R) and lack expression of CD45, and these markers can be used, e.g., by flow cytometry, to identify plasma cells. CD27 is also a good marker for plasma cells as naive B cells are CD27−, memory B cells are CD27+ and plasma cells are CD27++. Memory B cell subsets may also express surface IgG, IgM and IgD, whereas plasma cells do not express these markers on the cell surface. CD38 and CD138 are expressed at high levels on plasma cells (See, Jourdan et al. Blood. 2009 Dec. 10;114 (25): 5173-81; Trends Immunol. 2009 June; 30 (6): 277-285; Nature Reviews, 2005, 5:231-242; Nature Med. 2010, 16:123-129; Neuberger, M. S.; Honjo, T.; Alt, Frederick W. (2004). Molecular biology of B cells. Amsterdam: Elsevier, pp. 189-191; Bertil Glader; Greer, John G.; John Foerster; Rodgers, George G.; Paraskevas, Frixos (2008). Wintrobe's Clinical Hematology, 2-Vol. Set. Hagerstwon, MD: Lippincott Williams & Wilkins. pp. 347; Walport, Mark; Murphy, Kenneth; Janeway, Charles; Travers, Paul J. (2008). Janeway's immunobiology. New York: Garland Science, pp. 387-388; Rawstron AC (May 2006). “Immunophenotyping of plasma cells”. Curr Protoc Cytom).

Examples of modified differentiated B cells are described in WO2018201071, incorporated herein by reference.

As used herein, the term “differentiated B cell” is a cell that is differentiated towards the end of the B cell lineage. In some embodiments, the differentiated B cell is a mature B cell, a memory B cell, a plasmablast, or a plasma cell. Therefore, a differentiated B cell that has been transfected with a transgene would be a modified differentiated B cell.

In certain embodiments of the methods described herein, B cells can be obtained from a unit of blood collected from a subject using any number of techniques known to the skilled artisan, such as FICOLL™ (copolymers of sucrose and epichlorohydrin that may be used to prepare high density solutions) separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the methods described herein, the cells are washed with phosphate buffered saline (PBS). In an alternative embodiment, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, PBS. Alternatively, the undesirable components of the apheresis sample may be removed and the cells directly resuspended in culture media.

B cells may be isolated from peripheral blood or leukapheresis using techniques known in the art. For example, PBMCs may be isolated using FICOLL™ (Sigma-Aldrich, St Louis, MO) and CD19+ B cells purified by negative or positive selection using any of a variety of antibodies known in the art, such as the Rosette tetrameric complex system (StemCell Technologies, Vancouver, Canada) or MACS™ MicroBead Technology (Miltenyi Biotec, San Diego, CA). In certain embodiments, memory B cells are isolated as described by Jourdan et al., (Blood. 2009 Dec. 10; 114 (25): 5173-81). For example, after removal of CD2+ cells using anti-CD2 magnetic beads, CD19+ CD27+ memory B cells can be sorted by FACS. Bone marrow plasma cells (BMPCs) can be purified using anti-CD138 magnetic microbeads sorting or other similar methods and reagents.

Other isolation kits are commercially available, such as R&D Systems' MagCellect Human B Cell Isolation Kit (Minneapolis, MN). In certain embodiments, resting B cells may be prepared by sedimentation on discontinuous Percoll gradients, as described in (Defranco et al., (1982) J. Exp. Med. 155:1523).

In one embodiment, PBMCs are obtained from a blood sample using a gradient based purification (e.g., FICOLL™). In another embodiment, PBMCs are obtained from aphersis based collection. In one embodiment, B cells are isolated from PBMCs by isolating pan B cells. The isolating step may utilize positive and/or negative selection. In one embodiment, the negative selection comprises depleting T cells using anti-CD3 conjugated microbeads, thereby providing a T cell depleted fraction. In a further embodiment, memory B cells are isolated from the pan B cells or the T cell depleted fraction by positive selection for CD27.

In one embodiment, switched memory B cells are obtained. “Switched memory B cell” or “switched B cell,” as used herein, refers to a B cell that has undergone isotype class switching. In one embodiment, switched memory B cells are positively selected for IgG. In another embodiment, switched memory B cells are obtained by depleting IgD and IgM expressing cells.

In a further embodiment the promoter sequence from a gene unique to memory B cells, such as, e.g., the CD27 gene (or other gene specific to memory B cells and not expressed in naive B cells) is used to drive expression of a selectable marker such as, e.g., mutated dihydrofolate reductase allowing for positive selection of the memory B cells in the presence of methotrexate. In another embodiment T cells are depleted using CD3 or by addition of cyclosporin. In another embodiment, CD138+ cells are isolated from the pan B cells by positive selection. In yet another embodiment, CD138+ cells are isolated from PBMCs by positive selection. In another embodiment, CD38+ cells are isolated from the pan B cells by positive selection. In yet another embodiment, CD38+ cells are isolated from PBMCs by positive selection. In one embodiment, CD27+ cells are isolated from PBMCs by positive selection. In another embodiment, memory B cells and/or plasma cells are selectively expanded from PBMCs using in vitro culture methods available in the art.

Terminally differentiated plasma cells do not express common pan-B cell markers, such as CD19 and CD20, and express relatively few surface antigens. Plasma cells express CD38, CD78, CD138 and interleukin-6 receptor (IL-6R) and lack expression of CD45, and these markers can be used, e.g., by flow cytometry, to identify plasma cells. CD27 is also a good marker for plasma cells as naive B cells are CD27−, memory B cells are CD27+ and plasma cells are CD27++. Memory B cell subsets may also express surface IgG, IgM and IgD, whereas plasma cells do not express these markers on the cell surface. CD38 and CD138 are expressed at high levels on plasma cells (See: Jourdan et al. Blood. 2009 Dec. 10;114 (25): 5173-81; Trends Immunol. 2009 June; 30 (6): 277-285; Nature Reviews, 2005, 5:231-242; Nature Med. 2010, 16:123-129; Neuberger, M. S.; Honjo, T.; Alt, Frederick W. (2004). Molecular biology of B cells. Amsterdam: Elsevier, pp. 189-191; Bertil Glader; Greer, John G.; John Foerster; Rodgers, George G.; Paraskevas, Frixos (2008). Wintrobe's Clinical Hematology, 2-Vol. Set. Hagerstwon, MD: Lippincott Williams & Wilkins. pp. 347; Walport, Mark; Murphy, Kenneth; Janeway, Charles; Travers, Paul J. (2008). Janeway's immunobiology. New York: Garland Science, pp. 387-388; Rawstron AC (May 2006). “Immunophenotyping of plasma cells”. Curr Protoc Cytom).

The present disclosure describes a modified differentiated B cells capable of expressing at least one therapeutic protein and wherein the cells are capable of homing to bone marrow.

In some embodiments, the modified differentiated B cell expresses at least one therapeutic protein. In some embodiments, the modified differentiated B cell is CD38+. In some embodiments, the modified differentiated B cell is CD138+. In some embodiments, the modified differentiated B cell is CD78+. In some embodiments, the modified differentiated B cell is IL-6R+. In some embodiments, the modified differentiated B cell is CD27++. In some embodiments, the modified differentiated B cell is CD20−, wherein CD20− refers to low expression or down regulation. In some embodiments, the modified differentiated B cell is CD138^{high} (CD138++). In some embodiments, the modified differentiated B cell is CD38+, CD138+, CD78+, IL-6R+, and CD27++. In some embodiments, the modified differentiated B cell is CD38+, CD138+, CD78+, IL-6R+, CD27++, CD20−, and CD138^{high} (CD138++).

In some embodiments, the modified differentiated B cell expresses at least one therapeutic protein. In some embodiments, the modified differentiated B cell expresses one therapeutic protein. In some embodiments, the modified differentiated B cell expresses two therapeutic proteins. In some embodiments, the modified differentiated B cell expresses three therapeutic proteins. In some embodiments, the modified differentiated B cell expresses more than three therapeutic proteins.

In some embodiments, the modified differentiated B cell comprises a suicide gene. In some embodiments, the modified differentiated B cell comprises a selection marker. In some embodiments, the modified differentiated B cell is capable of expressing an exogenous enzyme. In some embodiments, the exogenous enzyme is a therapeutic enzyme. In some embodiments, the exogenous enzyme is an exogenous glycosyltransferase. In some embodiments, the exogenous glycosyltransferase is a beta-1,4-mannosylglycoprotein 4-beta-N-acetylglucosaminyltransferase (MGAT3).

In some embodiments, the at least one therapeutic protein is capable of binding at least one tumor associated antigen. In some embodiments, the at least one therapeutic protein is capable of binding at least two tumor associated antigens. In some embodiments, the at least one therapeutic protein is capable of binding at least one tumor associated antigens and at least one cell-specific or tissue-specific antigen.

In some embodiments, the at least one therapeutic protein is an exogenous antigen-specific antibody or an exogenous antigen-binding fragment thereof. In some embodiments, the at least one therapeutic protein is a bispecific antibody. In some embodiments, the at least one therapeutic protein is a chimeric antibody. In some embodiments, the antigen-binding fragment is a single-chain variable fragment (scFv). In some embodiments, the antigen-binding fragment is an F(ab′)₂. In some embodiments, the antigen-binding fragment is an Fab. In some embodiments, the antigen-binding fragment is an Fab′.

In some embodiments, wherein the therapeutic protein is an anti-PSMA antibody or an anti-PSMA antibody fragment thereof, or the therapeutic protein is an anti-HER-2 antibody or an anti-HER-2 antibody fragment thereof, or the therapeutic protein is an anti-MUC1 antibody or an anti-MUC1 antibody fragment thereof, or the therapeutic protein is an anti-NYESO-1 antibody or an anti-NYESO-1 antibody fragment thereof, or the therapeutic protein is an anti-CEA antibody or an anti-CEA antibody fragment thereof, or the therapeutic protein is an anti-MAGE-A1 antibody or an anti-MAGE-A1 antibody fragment thereof, or the therapeutic protein is an anti-α-fetoprotein antibody or an anti-α-fetoprotein antibody fragment thereof, or the therapeutic protein is an anti-CA 19-9 antibody or an anti-CA 19-9 antibody fragment thereof.

In some embodiments, the anti-PSMA antibody is 3/A12 or 3/A12 variant. In some embodiments, the anti-PSMA antibody fragment is a scFv. In some embodiments, the scFv is A5 scFv or a derivative of A5 scFv. In some embodiments, the scFv is conjugated to a IgG1Fc.

In some embodiments, the anti-PSMA antibody is J591 or J591 variant. In some embodiments, the anti-PSMA antibody fragment is a scFv. In some embodiments, the anti-PSMA antibody comprises a sequence that is 95%, 96%, 97%, 98%, or 99% identical to SEQ ID NO: 25. In some embodiments, the anti-PSMA antibody comprises or consists of the sequence set forth in SEQ ID NO: 25.

In some embodiments, the anti-HER-2 antibody, or anti-HER-2 antibody fragment thereof, is Herceptin or a Herceptin variant. In some embodiments, the anti-HER-2 antibody comprises a sequence that is 95%, 96%, 97%, 98%, or 99% identical to either SEQ ID NO: 9 or SEQ ID NO: 1. In some embodiments, the anti-HER-2 antibody comprises or consists of the sequence set forth in either SEQ ID NO: 9 or SEQ ID NO: 1.

In some embodiments, the therapeutic protein is any one of herceptin, enfortumab vedotin-ejfv, sacituzumab govitecan-hziy, ado-trastuzumab emtansine, fam-trastuzumab deruxtecan-nxki, margetuximab-cmkb, pertuzumab pertuzumab trastuzumab, and hyaluronidase-zzxf (Phesgo), sacituzumab govitecan-hziy, trastuzumab, tisotumab vedotin-tftv, panitumumab, iobenguane I 131, fam-trastuzumab deruxtecan-nxki, trastuzumab, denosumab, avelumab, alemtuzumab, blinatumomab, brexucabtagene autoleucel, gemtuzumab ozogamicin, inotuzumab ozogamicin, moxetumomab pasudotox-tdfk, obinutuzumab, ofatumumab, rituximab, rituximab and hyaluronidase human, tagraxofusp-erzs, tisagenlecleucel, atezolizumab, durvalumab, atezolizumab, fam-trastuzumab deruxtecan-nxki, necitumumab, ramucirumab, axicabtagene ciloleucel, brentuximab vedotin, brexucabtagene autoleucel, denileukin diftitox, ibritumomab tiuxetan, lisocabtagene maraleucel, loncastuximab tesirine-lpyl, mogamulizumab-kpkc, mosunetuzumab-axgb, obinutuzumab, polatuzumab vedotin-piiq, rituximab, rituximab and hyaluronidase human, siltuximab, tafasitamab-cxix, tisagenlecleucel, dinutuximab, naxitamab-gqgk, mirvetuximab soravtansine-gynx, nivolumab and relatlimab-rmbw, atezolizumab, fam-trastuzumab deruxtecan-nxki, ramucirumab, or trastuzumab.

In some embodiments, the exogenous antigen-specific antibody or an exogenous antigen-binding fragment thereof has a modified fragment crystallizable (Fc) region. In some embodiments, the modified fragment crystallizable (Fc) region comprises one or more glycan modifications. In some embodiments, the glycan modification is at asparagine 297 (Asn297) of the CH2 domain. In some embodiments, the modified fragment crystallizable (Fc) region comprises an afucosylated glycan at asparagine 297 of the CH2 domain. In some embodiments, the modified fragment crystallizable (Fc) region comprises a glycan that comprises a bisecting N-acetylglucosamine at asparagine 297 of the CH2 domain. In some embodiments, the CH2 sequence is set forth in SEQ ID NO: 7.

In some embodiments, the modified fragment crystallizable (Fc) region comprises a G236A mutation. In some embodiments, the modified fragment crystallizable (Fc) region comprises a S239D mutation. In some embodiments, the modified fragment crystallizable (Fc) region comprises a 1332E mutation. In some embodiments, the modified fragment crystallizable (Fc) region comprises a A330L mutation. In some embodiments, the modified fragment crystallizable (Fc) region comprises one or more of a G236A, S239D, 1332E, and A330L mutation. In some embodiments, the modified fragment crystallizable (Fc) region comprises a G236A, S239D, 1332E, and A330L mutation.

In some embodiments, the modified fragment crystallizable (Fc) region comprises a S267E mutation. In some embodiments, the modified fragment crystallizable (Fc) region comprises a H268F mutation. In some embodiments, the modified fragment crystallizable (Fc) region comprises a S324T mutation. In some embodiments, the modified fragment crystallizable (Fc) region comprises one or more of a S267E, H268F, and S324T mutation. In some embodiments, the modified fragment crystallizable (Fc) region a S267E, H268F, and S324T mutation

In some embodiments, the modified fragment crystallizable (Fc) region comprises one or more of a S267E, H268F, S324T, G236A, S239D, 1332E, and A330L mutation.

In some embodiments, the modified fragment crystallizable (Fc) region comprises a M428L mutation. In some embodiments, the modified fragment crystallizable (Fc) region comprises a N434S mutation. In some embodiments, the modified fragment crystallizable (Fc) region comprises a M252Y mutation. In some embodiments, the modified fragment crystallizable (Fc) region comprises a S254T mutation. In some embodiments, the modified fragment crystallizable (Fc) region comprises a T256E mutation. In some embodiments, the modified fragment crystallizable (Fc) region comprises a H433K mutation. In some embodiments, the modified fragment crystallizable (Fc) region comprises a N434F mutation. In some embodiments, the modified fragment crystallizable (Fc) region comprises one or more of a M428L, N434S, M252Y, S254T, T256E, H433K, and N434F mutation. In some embodiments, the modified fragment crystallizable (Fc) region comprises one or more of a S267E, H268F, S324T, G236A, S239D, 1332E, A330L, M428L, N434S, M252Y, S254T, T256E, H433K, and N434F mutation.

In some embodiments, the modified fragment crystallizable (Fc) region comprises a mutation to enhance Fc effector functions and/or increase the IgG half-life in vivo.

The antibodies described above, and other binding molecules may be used, for example, for identifying tissue which expresses a tumor-associated antigen. Antibodies may also be coupled to specific diagnostic substances for displaying cells and tissues expressing tumor-associated antigens. They may also be coupled to therapeutically useful substances. Diagnostic substances comprise, in a nonlimiting manner, barium sulfate, iocetamic acid, iopanoic acid, calcium ipodate, sodium diatrizoate, meglumine diatrizoate, metrizamide, sodium tyropanoate and radio diagnostics, including positron emitters such as fluorine-18 and carbon-11, gamma emitters such as iodine-123, technetium-99m, iodine-131 and indium-111, nuclides for nuclear magnetic resonance, such as fluorine and gadolinium. According to the invention, the term “therapeutically useful substance” means any therapeutic molecule which, as desired, is selectively guided to a cell which expresses one or more tumor-associated antigens, including anticancer agents, radioactive iodine-labeled compounds, toxins, cytostatic or cytolytic drugs, etc. Anticancer agents comprise, for example, aminoglutethimide, azathioprine, bleomycin sulfate, busulfan, carmustine, chlorambucil, cisplatin, cyclophosphamide, cyclosporine, cytarabidine, dacarbazine, dactinomycin, daunorubin, doxorubicin, taxol, etoposide, fluorouracil, interferon-α, lomustine, mercaptopurine, methotrexate, mitotane, procarbazine HCl, thioguanine, vinblastine sulfate and vincristine sulfate. Other anticancer agents are described, for example, in Goodman and Gilman, “The Pharmacological Basis of Therapeutics”, 8th Edition, 1990, McGraw-Hill, Inc., in particular Chapter 52 (Antineoplastic Agents (Paul Calabresi and Bruce A. Chabner). Toxins may be proteins such as pokeweed antiviral protein, cholera toxin, pertussis toxin, ricin, gelonin, abrin, diphtheria exotoxin or Pseudomonas exotoxin. Toxin residues may also be high energy-emitting radionuclides such as cobalt-60.

In some embodiments, the therapeutic protein is expressed from a transgene encoded within a transposon. In some embodiments, the transposon is a Sleeping Beauty transposon. In some embodiments, the therapeutic protein is expressed from a transgene encoded in a DNA or mRNA.

In some embodiments, the transgene is incorporated into the differentiated B cell genome. In some embodiments, the transgene is incorporated into the immunoglobulin heavy chain locus. In some embodiments, the transgene is incorporated by a transposase, integrase, or recombinase.

In some embodiments, the therapeutic protein is anchored to the differentiated B cell membrane. In some embodiments, the therapeutic protein is secreted.

In some embodiments, the modified differentiated B cell secretes one or more of a cytokine, a signaling molecule, an enzyme, a protein, a peptide, a DNA, a RNA, or a small molecule.

In some embodiments, the modified differentiated B cells are capable of increased CXCR4 expression.

### Manufacture of B Cell Compositions

Provided herein are methods to produce differentiated B cell populations. In typical embodiments, the method comprises (a) isolating pan-B cells, memory B cells, switch memory B cells, plasmablasts, or plasma cells from a sample, thereby obtaining an isolated B cell population; (b) culturing the isolated B cell population in vitro with one or more B cell activating factors, thereby obtaining an expanded B cell population; (c) transfecting the expanded B cell population with a transgene; and (d) differentiating the expanded B cell population in vitro with one or more B cell activating factors, thereby obtaining a modified differentiated B cell population. Further examples and embodiments are described in WO2018201071, incorporated herein by reference.

In some embodiments, the transfecting step comprises enriching the B cell population using a selectable marker. In some embodiments, the selectable marker is selected from the group consisting of a fluorescent marker protein, a drug resistance factor, and a surface marker. In some embodiments, the surface marker is a truncated epidermal growth factor receptor marker, i.e., a EGFRt marker. In some embodiments, EGFRt facilitates in vivo detection of the administered, transduced B cells and, if the administered B cells cause unacceptable side effects, can promote elimination of those cells through a cetuximab-induced antibody-dependent cellular cytotoxicity (ADCC) response.

In some embodiments, the transfecting step comprises electroporation, lipofection, non-viral transduction, or viral transduction. In some embodiments, the transfecting step comprises mechanical delivery or ultrasonic delivery. In some embodiments, the non-viral vector is a transposon. In some embodiments, the transfecting step comprises AAV or lentivirus. In some embodiments, the transposon is a sleeping beauty transposon.

In some embodiments, the transposon comprises an EEK promoter. In some embodiments, the transposon comprises an EFla promoter. In some embodiments, the transposon comprises both an EEK promoter and an EFla promoter. In some embodiments, the transposon comprises an IRES or ribosome skipping or slipping sequence. In some embodiments, the transposon comprises a bicistronic system. In some embodiments, the bicistronic system is expressed from an EEK promoter or an EF1α promoter.

In some embodiments, the bicistronic system comprises an immunoglobulin heavy chain and an immunoglobulin light chain. In some embodiments, the bicistronic system comprises an immunoglobulin heavy chain, an immunoglobulin light chain, and EGFRt.

In some embodiments, the bicistronic system comprises an immunoglobulin heavy chain, an immunoglobulin light chain, and EGFRt, wherein the immunoglobulin heavy chain and immunoglobulin light chain are expressed from an EFla or EEK promoter. In some embodiments, the bicistronic system comprises an EGFRt, wherein the EGFRt is expressed from an EEK or EFla promoter. In some embodiments, the bicistronic system comprises an immunoglobulin heavy chain, an immunoglobulin light chain, and EGFRt, wherein the immunoglobulin heavy chain and immunoglobulin light chain are expressed from an EEK promoter and wherein the EGFRt is expressed an EF1 α promoter.

In some embodiments, an antibody or an antibody fragment is expressed from an EEK promoter or an EF1α promoter. In some embodiments, a scFv is expressed from an EEK promoter or an EF1α promoter. In some embodiments, the scFv is an anti-HER-2 antibody. In some embodiments, the scFv is an anti-PSMA antibody. In some embodiments, the scFv comprises a sequence that set forth in SEQ ID NO: 1 or 25.

In some embodiments, the transposon comprises one or more transgenes. In some embodiments, the transgene encodes at least one therapeutic protein. In some embodiments, the therapeutic protein is an anti-PSMA antibody or an anti-PSMA antibody fragment thereof, or the therapeutic protein is an anti-HER-2 antibody or an anti-HER-2 antibody fragment thereof, or the therapeutic protein is an anti-MUCI antibody or an anti-MUC1 antibody fragment thereof, or the therapeutic protein is an anti-NYESO-1 antibody or an anti-NYESO-1 antibody fragment thereof, or the therapeutic protein is an anti-CEA antibody or an anti-CEA antibody fragment thereof, or the therapeutic protein is an anti-MAGE-A1 antibody or an anti-MAGE-A1 antibody fragment thereof, or the therapeutic protein is an anti-α-fetoprotein antibody or an anti-α-fetoprotein antibody fragment thereof, or the therapeutic protein is an anti-CA 19-9 antibody or an anti-CA 19-9 antibody fragment thereof.

In some embodiments, the anti-PSMA therapeutic protein is an antigen-specific antibody or a scFv or an antibody fragment thereof.

In some embodiments, the anti-PSMA therapeutic protein is a 3/A12 or a variant of 3/A12. In some embodiments, the anti-PSMA therapeutic protein is an A5 scFv or a variant of A5. In some embodiments, the anti-PSMA therapeutic protein is a J591 or a variant of J591. In some embodiments, sequence of the anti-PSMA therapeutic protein is set forth in SEQ ID NO: 25.

In some embodiments, the anti-HER therapeutic protein is Herceptin or Herceptin fragment thereof. In some embodiments, sequence of the anti-HER therapeutic protein is set forth in SEQ ID NO: 1.

In some embodiments, the therapeutic protein further comprises an epitope tag. In some embodiments, the epitope tag is a HIS-tag, or a MYC-tag, or a HA-tag.

In some embodiments, the transgene incorporates into the B cell genome. In some embodiments, the transgene incorporates into the heavy chain locus or the light chain locus or both.

In some embodiments, the modified differentiated B cell is capable of expressing a cytokine, a signaling molecule, or a small molecule.

### Administration and Treatment

Accordingly, the methods for administering modified differenatiated B cell compositions described herein are useful for long term in vivo delivery and expression of therapeutic agents. The present disclosure relates generally to methods for achieving sufficient enrichment and number of cells producing a therapeutic agent and sufficient levels of the therapeutic agent in vivo.

Described herein is a method of treating a subject having a cancer that has metastasized to the bone marrow comprising administering to the subject a therapeutically effective amount of the population of modified differentiated B cells. In typical embodiments, the modified differentiated B cells express at least one therapeutic protein.

In certain embodiments, the cancer is prostate cancer, breast cancer, lung cancer, brain cancer, kidney cancer, skin cancer, multiple myeloma, thyroid cancer, stomach cancer, lymphoma, leukemia, bone cancer, cervical cancer, ovarian cancer, bladder cancer, eye cancer, testicular cancer, pancreatic cancer, liver and bile duct cancer, malignant mesothelioma, myelodysplastic and myeloproliferative cancer, fallopian tube cancer, primary peritoneal cancer, plexiform neurofibroma, a solid tumor, gastric cancer, systemic mastocytosis, or sarcoma. In typical embodiments, the cancer has metastasized to the bone. In typical embodiments, the modified differentiated B cells are capable of navigating or homing to the bone. In some embodiments, treatment with the modified differentiated B cells reduce the cancer or tumor burden in the bone. In some embodiments, treatment with the modified differentiated B cells eliminate the cancer or tumor burden in the bone.

In some embodiments, the cancer is PSMA positive. In some embodiments, the cancer is HER2 positive. In some embodiments, the cancer is CD20 positive. In some embodiments, the cancer is CD33 positive. In some embodiments, the cancer is CD22 positive. In some embodiments, the cancer is Nectin-4 positive. In some embodiments, the cancer is TROP2 positive. In some embodiments, the cancer is CD19 positive. In some embodiments, the cancer is PD-L1 positive. In some embodiments, the cancer is PD-1 positive. In some embodiments, the cancer is EGFR positive. In some embodiments, the cancer is GD2 positive. In some embodiments, the cancer is CD38 positive. In some embodiments, the cancer is CCR4 positive. In some embodiments, the cancer is CD30 positive. In some embodiments, the cancer is tissue factor positive. In some embodiments, the cancer is NE transporter positive. In some embodiments, the cancer is CD52 positive. In some embodiments, the cancer is CD123 positive. In some embodiments, the cancer is VEGF OR VEGF2 positive. In some embodiments, the cancer is CCR4 positive. In some embodiments, the cancer is CD79B positive. In some embodiments, the cancer is IL6-R positive. In some embodiments, the cancer is folate RA positive. In some embodiments, the cancer is PD-1 and LAG3 positive. In some embodiments, the cancer is BCMA positive. In some embodiments, the cancer is IL2-R positive. In some embodiments, the cancer is RANKL positive. In some embodiments, the cancer is positive for one or more of the above listed markers.

In typical embodiments, the cancer is in the bone. In some embodiments, the cancer is metastatic cancer. In typical embodiments, the modified differentiated B cells are capable of navigating or homing to the bone.

Long term survival may be measured in days, weeks, or even years. In one embodiment, a majority of the modified differentiated B cells survive in vivo for 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more days post-administration. In one embodiment, a majority of the modified differentiated B cells survive in vivo for 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or more weeks post-administration. In another embodiment, the modified differentiated B cells survive in vivo for 1, 1.5, 2, 2.5, 3, 3.5, 4, 4.5, 5, 6, 7, 8, 9, 10, 11, 12, 13, 14, 15, 16, 17, 18, 19, 20, 25, 30 or more years. Additionally, while the modified differentiated B cells described herein may survive in vivo for 10 or more days, it is understood that a majority of the modified differentiated B cells survive in vivo for 1, 2, 3, 4, 5, 6, 7, 8, 9 or more days post-administration. Accordingly, it is contemplated that modified differentiated B cells described herein are useful for short-term treatment (e.g., 4 days) and long-term treatment (e.g., 30 or more days) methods.

In some embodiments, the modified differentiated B cell has the capacity to engraft into a tissue or location. In some embodiments, the modified differentiated B cell is capable of engraftment into the bone.

In some embodiments, the engrafted differentiated B cell is stable for greater than 1 month. In some embodiments, the engrafted differentiated B cell is stable for greater than 2 months, or 3 months, or 4 months, or 5 months, or 6 months, or 7 months, or 8 months, or 9 months, or 10 months, or 11 months, or 12 months. In some embodiments, the engrafted differentiated B cell is stable for greater than 1 year.

The disclosure herein describes a method of delivering a genetically modified differentiated B cell to the central nervous system of a human subject comprising administering one or more doses of the modified differentiated B cell to a subject's periphery, wherein the periphery comprises vasculature, muscle, or bone of the subject.

In certain embodiments, the administration step comprises intravenous injection, or intraarterial injection, or intramuscular injection, or intraosseous injection. In some embodiments, the administration step comprises intracerebroventricular injection.

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the B cell compositions of the present disclosure are administered to a patient by intradermal or subcutaneous injection. In another embodiment, the B cell compositions as described herein are preferably administered by i.v. injection. The compositions of B cells may be injected directly into a tumor, lymph node, bone marrow or site of infection.

In yet another embodiment, the pharmaceutical composition can be delivered in a controlled release system. In one embodiment, a pump may be used (see Langer, 1990, Science 249:1527-1533; Sefton 1987, CRC Crit. Ref. Biomed. Eng. 14:201; Buchwald et al., 1980; Surgery 88:507; Saudek et al., 1989, N. Engl. J. Med. 321:574). In another embodiment, polymeric materials can be used (see Medical Applications of Controlled Release, 1974, Langer and Wise (eds.), CRC Pres., Boca Raton, Fla.; Controlled Drug Bioavailability, Drug Product Design and Performance, 1984, Smolen and Ball (eds.), Wiley, New York; Ranger and Peppas, 1983; J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled release system can be placed in proximity of the therapeutic target, thus requiring only a fraction of the systemic dose (see, e.g., Medical Applications of Controlled Release, 1984, Langer and Wise (eds.), CRC Pres., Boca Raton, Fla., vol. 2, pp. 1 15-138).

The differentiated B cell compositions of the present disclosure may also be administered using any number of matrices. Matrices have been utilized for a number of years within the context of tissue engineering (see, e.g., Principles of Tissue Engineering (Lanza, Langer, and Chick (eds.)), 1997. The present disclosure utilizes such matrices within the novel context of acting as an artificial lymphoid organ to support and maintain the differentiated B cells. Accordingly, the present disclosure can utilize those matrix compositions and formulations which have demonstrated utility in tissue engineering. Accordingly, the type of matrix that may be used in the compositions, devices and methods of the disclosure is virtually limitless and may include both biological and synthetic matrices. In one particular example, the compositions and devices set forth by U.S. Pat. Nos: 5,980,889; 5,913,998; 5,902,745; 5,843,069; 5,787,900; or 5,626,561 are utilized. Matrices comprise features commonly associated with being biocompatible when administered to a mammalian host. Matrices may be formed from natural and/or synthetic materials. The matrices may be nonbiodegradable in instances where it is desirable to leave permanent structures or removable structures in the body of an animal, such as an implant; or biodegradable. The matrices may take the form of sponges, implants, tubes, telfa pads, fibers, hollow fibers, lyophilized components, gels, powders, porous compositions, or nanoparticles. In addition, matrices can be designed to allow for sustained release seeded cells or produced cytokine or other active agent. In certain embodiments, the matrix of the present disclosure is flexible and elastic, and may be described as a semisolid scaffold that is permeable to substances such as inorganic salts, aqueous fluids and dissolved gaseous agents including oxygen.

A matrix is used herein as an example of a biocompatible substance. However, the current disclosure is not limited to matrices and thus, wherever the term matrix or matrices appears these terms should be read to include devices and other substances which allow for cellular retention or cellular traversal, are biocompatible, and are capable of allowing traversal of macromolecules either directly through the substance such that the substance itself is a semi-permeable membrane or used in conjunction with a particular semi-permeable substance.

In certain embodiments of the present disclosure, differentiated B cells transfected and activated using the methods described herein, or other methods known in the art, are administered to a patient in conjunction with (e.g. before, simultaneously or following) any number of relevant treatment modalities, including but not limited to treatment with agents such as antiviral agents, chemotherapy, radiation, immunosuppressive agents, such as cyclosporin, bisulfin, bortezomib, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506), the proteasome (bortezomib), or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993; Isoniemi (supra)). In a further embodiment, the cell compositions of the present disclosure are administered to a patient in conjunction with (e.g. before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In one embodiment, the cell compositions of the present disclosure are administered following B-cell ablative therapy such as agents that react with CD20, e.g. Rituxan®. In one embodiment, the cell compositions of the present disclosure are administered following B cell ablative therapy using an agent such as bortezomib. For example, in one embodiment, subjects may undergo standard treatment with high dose chemotherapy followed by peripheral blood stem cell transplantation. In certain embodiments, following the transplant, subjects receive an infusion of the expanded immune cells of the present disclosure. In an additional embodiment, expanded cells are administered before or following surgery.

The disclosure herein describes a method of treating or preventing a human disease or disorder in a subject in need thereof. In certain embodiments, the method of treating or preventing a human disease in a subject in need thereof comprises administering a therapeutically effective amount of a genetically modified differentiated B cell. In typical embodiments, the subject has or is suspected of having a disease or disorder associated with cancer.

In one embodiment, a single dose of modified differentiated B cells is administered to a subject. In one embodiment, two or more doses of modified differentiated B cells are administered sequentially to a subject. In one embodiment, three doses of modified differentiated B cells are administered sequentially to a subject. In one embodiment, a dose of modified differentiated B cells is administered weekly, biweekly, monthly, bimonthly, quarterly, semiannually, annually, or biannually to a subject. In one embodiment, a second or subsequent dose of modified differentiated B cells is administered to a subject when an amount of a therapeutic agent produced by the modified differentiated B cells decreases.

In one embodiment, a dose of modified differentiated B cells is administered to a subject at a certain frequency (e.g., weekly, biweekly, monthly, bimonthly, or quarterly) until a desired amount (e.g., an effective amount) of a therapeutic agent is detected in the subject. In one embodiment, an amount of the therapeutic agent is monitored in the subject. In one embodiment, a subsequent dose of modified differentiated B cells is administered to the subject when the amount of the therapeutic agent produced by the modified differentiated B cells decreases below the desired amount. In one embodiment, the desired amount is a range that produces the desired effect.

When “an effective amount,” “an anti-tumor effective amount,” “a tumor-inhibiting effective amount,” or “therapeutic amount” is indicated, the precise amount of the compositions of the present disclosure to be administered can be determined by a physician with consideration of individual differences in age, weight, tumor size, extent of infection or metastasis, and condition of the patient (subject). Differentiated B cell compositions may also be administered multiple times at an appropriate dosage(s). The cells can be administered by using infusion techniques that are commonly known in immunotherapy (see, e.g., Rosenberg et al., New Eng. J. of Med. 319:1676, 1988).

The optimal dosage and treatment regime for a particular patient can be determined by one skilled in the art of medicine by monitoring the patient for signs of disease and adjusting the treatment accordingly. The treatment may also be adjusted after measuring the levels of a therapeutic agent (e.g., a gene or protein of interest) in a biological sample (e.g., body fluid or tissue sample) can also be used to assess the treatment efficacy, and the treatment may be adjusted accordingly to increase or decrease. Typically, in related adoptive immunotherapy studies, antigen-specific T cells are administered approximately at 2×10⁹ to 2×10¹¹ cells to the patient. (See, e.g., U.S. Pat. No. 5,057,423).

In some aspects of the present disclosure, lower numbers of the modif differentiated B cells of the present disclosure, in the range of 10⁶/kilogram (10⁶-10¹¹ per patient) may be administered. In certain embodiments, the differentiated B cells are administered at 1×10⁴, 5×10⁴, 1×10⁵, 5×10⁵, 1×10⁶, 5×10⁶, 1×10⁷, 5×10⁷, 1×10⁸, 5×10⁸, 5×10⁹, 1×10¹⁰, 5×10¹⁰, 1×10¹¹, 5×10¹¹, or 1×10¹² cells to the subject. Differentiated B cell compositions may be administered multiple times at dosages within these ranges. The cells may be autologous or heterologous (e.g., allogeneic) to the patient undergoing therapy. If desired, the treatment may also include administration of mitogens (e.g., PHA) or lymphokines, cytokines, and/or chemokines (e.g., GM-CSF, IL-4, IL-13, Flt3-L, RANTES, MIP1, etc.) as described herein to enhance induction of an immune response and engraftment of the infused B cells.

The disclosure herein describes a method of manufacturing an engineered or modified differentiated B cell, wherein the B cell has a migratory capacity across the blood brain barrier (BBB) and secretes a therapeutic protein, the method comprising (i) obtaining and isolating immune cells from the blood of a subject; introducing to the cells an exogeneous polynucleotide encoding the therapeutic protein and an exogeneous polynucleotide encoding CCR6 and/or CCR7 cytokine receptors; (iii) selecting cells by one or more selection markers, wherein the selected cells express both the therapeutic protein and CCR6 and/or CCR7 cytokine receptors; (iv) expanding the selected cells ex vivo to produce and expanded population of cells; and (v) differentiating the expanded cells ex vivo into plasma cells and/or plasmablasts.

### Culturing B Cells in Vitro

B cells, such as memory B cells, can be cultured using in vitro methods to activate and differentiate the B cells into plasma cells or plasmablasts or both. As would be recognized by the skilled person, plasma cells may be identified by cell surface protein expression patterns using standard flow cytometry methods. For example, terminally differentiated plasma cells express relatively few surface antigens, and do not express common pan-B cell markers, such as CD19 and CD20. Instead, plasma cells may be identified by expression of CD38, CD78, CD138, and IL-6R and lack of expression of CD45. CD27 may also be used to identify plasma cells as naïve B cells are CD27−, memory B cells are CD27+ and plasma cells are CD27++. Plasma cells express high levels of CD38 and CD138.

In one embodiment, the differentiated B cells are CD20−, CD38−, CD138− memory B cells. In one embodiment, the differentiated B cells are CD20−, CD38+, CD138+ plasma cells. In one embodiment, the differentiated B cells are activated and have a cell surface phenotype of CD20− CD38− CD138− CD27+. In typical embodiments, CD20− refers to low expression and/or down regulation of CD20−.

In one embodiment, the B cells are contacted with one or more B cell activating factors, e.g., any of a variety of cytokines, growth factors or cell lines known to activate and/or differentiate B cells (see e.g., Fluckiger, et al. Blood 1998 92:4509-4520; Luo, et al., Blood 2009 1 13:1422-1431). Such factors may be selected from the group consisting of, but not limited to, IL-1, IL-2, IL-3, IL-4, IL-5, IL-6, IL-7, IL-8, IL-9, IL-10, IL-1 1, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, and IL-35, IFN-γ, IFN-α, IFN-β, IFN-δ, C type chemokines XCL1 and XCL2, C—C type chemokines (to date including CCL1-CCL28) and CXC type chemokines (to date including CXCL1-CXCL17), and members of the TNF superfamily (e.g., TNF-α, 4-1 BB ligand, B cell activating factor (BLyS), FAS ligand, sCD40L (including multimeric versions of sCD40L; e.g., histidine-tagged soluble recombinant CD40L in combination with anti-poly-histidine mAb to group multiple sCD40L molecules together), Lymphotoxin, OX40L, RANKL, TRAIL), CpG, and other toll like receptor agonists (e.g., CpG).

B cell activating factors may be added to in vitro cell cultures at various concentrations to achieve the desired outcome (e.g., expansion or differentiation). In one embodiment, a B cell activating factor is utilized in expanding the B cells in culture. In one embodiment, a B cell activating factor is utilized in differentiating the B cells in culture. In another embodiment, the B cell activating factor is utilized in both expanding and differentiating the B cells in culture. In one embodiment, the B cell activating factor is provided at the same concentration for expanding and differentiating. In another embodiment, the B cell activating factor is provided at a first concentration for expanding and at a second concentration for differentiating. It is contemplated that a B cell activating factor may be 1) utilized in expanding the B cells and not in differentiating the B cells, 2) utilized in differentiating the B cells and not in expanding the B cells, or 3) utilized in expanding and differentiating the B cells.

For example, B cells are cultured with one or more B cell activating factors selected from CD40L, IL-2, IL-4, and IL-10 for expansion of the B cells. In one embodiment, the B cells are cultured with 0.25-5.0 μg/ml CD40L. In one embodiment, the concentration of CD40L is 0.5 μg/ml. In one embodiment a crosslinking agent (such as an anti-HIS antibody in combination with HIS-tagged CD40L) is used to create multimers of CD40L. In one embodiment molecules of CD40L are covalently linked or are held together using protein multimerization domains (e.g., the Fc region of an IgG or a leucine zipper domain). In one embodiment CD40L is conjugated to beads. In one embodiment CD40L is expressed from feeder cells. In one embodiment, the B cells are cultured with 1-10 ng/ml IL-2. In one embodiment, the concentration of IL-2 is 5 ng/ml. In one embodiment, the B cells are cultured with 1-10 ng/ml IL-4. In one embodiment, the concentration of IL-4 is 2 ng/ml. In one embodiment, the B cells are cultured with 10-100 ng/ml IL-10. In one embodiment, the concentration of IL-10 is 40 ng/ml.

In one embodiment, B cells are cultured with one or more B cell activating factors selected from CD40L, IL-2, IL-4, IL-10, IL-15 and IL-21 for expansion of the B cells. In one embodiment, the B cells are cultured with 0.25-5.0 μg/ml CD40L. In one embodiment, the concentration of CD40L is 0.5 μg/ml. In one embodiment a crosslinking agent (such as an anti-HIS antibody in combination with HIS-tagged CD40L) is used to create multimers of CD40L. In one embodiment molecules of CD40L are covalently linked or are held together using protein multimerization domains (e.g., the Fc region of an IgG or a leucine zipper domain). In one embodiment CD40L is conjugated to beads. In one embodiment CD40L is expressed from feeder cells. In one embodiment, the B cells are cultured with 1-10 ng/ml IL-2. In one embodiment, the concentration of IL-2 is 5 ng/ml. In one embodiment, the B cells are cultured with 1-10 ng/ml IL-4. In one embodiment, the concentration of IL-4 is 2 ng/ml. In one embodiment, the B cells are cultured with 10-100 ng/ml IL-10. In one embodiment, the concentration of IL-10 is 40 ng/ml. In one embodiment, the B cells are cultured with 50-150 ng/ml IL-15. In one embodiment, the concentration of IL-15 is 100 ng/ml. In one embodiment, the B cells are cultured with 50-150 ng/ml IL-21. In one embodiment, the concentration of IL-21 is 100 ng/ml. In a particular embodiment, the B cells are cultured with CD40L, IL-2, IL-4, IL-10, IL-15 and IL-21 for expansion of the B cells.

In another example, B cells are cultured with one or more B cell activating factors selected from CD40L, IFN-α and IFN-δmix, IL-2, IL-6, IL-10, IL-15, IL-21, and P-class CpG oligodeoxynucleotides (p-ODN) for differentiation of the B cells. In one embodiment, the B cells are cultured with 25-75 ng/ml CD40L. In one embodiment, the concentration of CD40L is 50 ng/ml. In one embodiment, the B cells are cultured with 250-750 U/ml IFN-α and IFN-δ mix. In one embodiment the concentration of the IFN-α and IFN-δ mix is 500 U/ml. In one embodiment, the B cells are cultured with 5-50 U/ml IL-2. In one embodiment the concentration of IL-2 is 20 U/ml. In one embodiment, the B cells are cultured with 25-75 ng/ml IL-6. In one embodiment, the concentration of IL-6 is 50 ng/ml. In one embodiment, the B cells are cultured with 10-100 ng/ml IL-10. In one embodiment, the concentration of IL-10 is 50 ng/ml. In one embodiment, the B cells are cultured with 1-20 ng/ml IL-15. In one embodiment, the concentration of IL-15 is 10 ng/ml. In one embodiment, the B cells are cultured with 10-100 ng/ml IL-21. In one embodiment, the concentration of IL-21 is 50 ng/ml. In one embodiment, the B cells are cultured with 1-50 μg/ml p-ODN. In one embodiment, the concentration of p-ODN is 10 μg/ml.

In one embodiment, B cells are contacted or cultured on feeder cells. In one embodiment, the feeder cells are a stromal cell line, e.g., murine stromal cell line S17 or MS5. In another embodiment, isolated CD19+ cells are cultured with one or more B cell activating factor cytokines, such as IL-10 and IL-4, in the presence of fibroblasts expressing CD40-ligand (CD40L, CD154). In one embodiment, CD40L is provided bound to a surface such as tissue culture plate or a bead. In another embodiment, purified B cells are cultured, in the presence or absence of feeder cells, with CD40L and one or more cytokines or factors selected from IL-10, IL-4, IL-7, p-ODN, CpG DNA, IL-2, IL-15, IL6, IFN-α, and IFN-δ.

In another embodiment, B cell activating factors are provided by transfection into the B cell or other feeder cell. In this context, one or more factors that promote differentiation of the B cell into an antibody secreting cell and/or one or more factors that promote the longevity of the antibody producing cell may be used. Such factors include, for example, Blimp-1, TRF4, anti-apoptotic factors like Bcl-xl or Bcl5, or constitutively active mutants of the CD40 receptor. Further, factors which promote the expression of downstream signaling molecules such as TNF receptor-associated factors (TRAFs) may also be used in the activation/differentiation of the B cells. In this regard, cell activation, cell survival, and antiapoptotic functions of the TNF receptor superfamily are mostly mediated by TRAF1-6 (see e.g., R. H. Arch, et al., Genes Dev. 12 (1998), pp. 2821-2830). Downstream effectors of TRAF signaling include transcription factors in the NF-κB and AP-1 family which can turn on genes involved in various aspects of cellular and immune functions. Further, the activation of NF-κB and AP-1 has been shown to provide cells protection from apoptosis via the transcription of antiapoptotic genes.

In another embodiment, Epstein Barr virus (EBV)-derived proteins are used for the activation and/or differentiation of B cells or to promote the longevity of the antibody producing cell. EBV-derived proteins include but are not limited to, EBNA-1, EBNA-2, EBNA-3, LMP-1, LMP-2, EBER, miRNAs, EBV-EA, EBV-MA, EBV-VCA and EBV-AN.

In certain embodiments, contacting the B cells with B cell activation factors using the methods provided herein leads to, among other things, cell proliferation (i.e., expansion), modulation of the lgM+ cell surface phenotype to one consistent with an activated mature B cell, secretion of Ig, and isotype switching. CD19+ B cells may be isolated using known and commercially available cell separation kits, such as the MiniMACS™ cell separation system (Miltenyi Biotech, Bergisch Gladbach, Germany). In certain embodiments, CD40L fibroblasts are irradiated before use in the methods described herein. In one embodiment, B cells are cultured in the presence of one or more of IL-3, IL-7, Flt3 ligand, thrombopoietin, SCF, IL-2, IL-10, G-CSF and CpG. In certain embodiments, the methods include culturing the B cells in the presence of one or more of the aforementioned factors in conjunction with transformed stromal cells (e.g., MS5) providing a low level of anchored CD40L and/or CD40L bound to a plate or a bead.

As discussed above, B cell activating factors induce expansion, proliferation, or differentiation of B cells. Accordingly, B cells are contacted with one or more B cell activating factors listed above to obtain an expanded cell population. A cell population may be expanded prior to transfection. Alternatively, or additionally, a cell population may be expanded following transfection. In one embodiment, expanding a B cell population comprises culturing cells with IL-2, IL-4, IL-10 and CD40L (see e.g., Neron et al. PLOS ONE, 2012 7 (12): e51946). In one embodiment, expanding a B cell population comprises culturing cells with IL-2, IL-10, CpG, and CD40L.

In another embodiment, expansion of a B cell population is induced and/or enhanced by a transgene introduced into the B cells. For example, a B cell that contains a recombinant receptor or an engineered receptor that induces a cell signaling pathway (e.g., signaling downstream of CD40) upon binding its ligand (e.g., a soluble ligand or a cell surface expressed ligand). In one embodiment, a B cell overexpresses CD40 due to expression of a CD40 transgene. In another embodiment, a B cell expresses an engineered receptor, including, e.g., a recombinantly engineered antibody. In one embodiment, an engineered receptor is similar to a chimeric antigen receptor (CAR) and comprises a fusion protein of an scFv and an intracellular signaling portion of a B cell receptor (e.g., CD40).

In one embodiment, expansion of a B cell population is induced and/or enhanced by a small molecule compound added to the cell culture. For example, a compound that binds to and dimerizes CD40 can be used to trigger the CD40 signaling pathway.

Any of a variety of culture media may be used in the present methods as would be known to the skilled person (see e.g., Current Protocols in Cell Culture, 2000-2009 by John Wiley & Sons, Inc.). In one embodiment, media for use in the methods described herein includes, but is not limited to Iscove modified Dulbecco medium (with or without fetal bovine or other appropriate serum). Illustrative media also includes, but is not limited to, IMDM, RPMI 1640, AIM-V, DMEM, MEM, a-MEM, F-12, X-Vivo 15, and X-Vivo 20. In further embodiments, the medium may comprise a surfactant, an antibody, plasmanate or a reducing agent (e.g. N-acetyl-cysteine, 2-mercaptoethanol), one or more antibiotics, and/or additives such as insulin, transferrin, sodium selenite and cyclosporin. In some embodiments, IL-6, soluble CD40L, and a cross-linking enhancer may also be used.

B cells are cultured under conditions and for sufficient time periods to achieve differentiation and/or activation desired. In certain embodiments, the B cells are cultured under conditions and for sufficient time periods such that 10%, 15%, 20%, 25%, 30%, 35%, 40%, 45%, 50%, 55%, 60%, 65%, 70%, 75%, 80%, 85%, 90%, 95%, or even 100% of the B cells are differentiated and/or activated as desired. In one embodiment, the B cells are activated and differentiated into a mixed population of plasmablasts and plasma cells. As would be recognized by the skilled person, plasmablasts and plasma cells may be identified by cell surface protein expression patterns using standard flow cytometry methods as described elsewhere herein, such as expression of one or more of CD38, CD78, IL-6R, CD27^{high}, and CD138 and/or lack of, or reduction of, expression of one or more of CD19, CD20 and CD45. As would be understood by the skilled person, memory B cells are generally CD20+ CD19+ CD27+ CD38− while early plasmablasts are CD20− CD19+ CD27++ CD38++. In one embodiment, the cells cultured using the methods described herein are CD20−, wherein CD20− refers to low expression, CD38+, CD138−. In another embodiment, the cells have a phenotype of CD20−, CD38+, CD138+. In certain embodiments, cells are cultured for 1-7 days. In further embodiments, cells are cultured 7, 14, 21 days or longer. Thus, cells may be cultured under appropriate conditions for 1, 2, 3, 4, 5, 6, 7, 8, 9, 10, 11, 12, 13, 14, 15, 16, 17, 18, 19, 20, 21, 22, 23, 24, 25, 26, 27, 28, 29, or more days. Cells are re-plated, and media and supplements may be added or changed as needed using techniques known in the art.

In certain embodiments, the B cells are cultured under conditions and for sufficient time periods such that at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100% of the cells are differentiated and activated to produce Ig and/or to express the transgene.

The induction of B cell activation may be measured by techniques such as ³H-uridine incorporation into RNA (as B cells differentiate, RNA synthesis increases), or by ³H-thymidine incorporation, which measures DNA synthesis associated with cell proliferation. In one embodiment, interleukin-4 (IL-4) may be added to the culture medium at an appropriate concentration (e.g., about 10 ng/ml) for enhancement of B cell proliferation.

Alternatively, B cell activation is measured as a function of immunoglobulin secretion. For example, CD40L is added to resting B cells together with IL-4 (e.g., 10 ng/ml) and IL-5 (e.g., 5ng/ml) or other cytokines that activate B cells. Flow cytometry may also be used for measuring cell surface markers typical of activated B cells. See e.g., Civin C I, Loken M R, Int'l J. Cell Cloning 987; 5:1-16; Loken, MR, et al, Flow Cytometry Characterization of Erythroid, Lymphoid and Monomyeloid Lineages in Normal Human Bone Marrow, in Flow Cytometry in Hematology, Laerum OD, Bjerksnes R. eds., Academic Press, New York 1992; pp. 31-42; and LeBein TW, et ai, Leukemia 1990; 4:354-358.

After culture for an appropriate period of time, such as from 2, 3, 4, 5, 6, 7, 8, 9, or more days, generally around 3 days, an additional volume of culture medium may be added. Supernatant from individual cultures may be harvested at various times during culture and quantitated for IgM and IgG1 as described in Noelle et al., (1991) J. Immunol. 146:1118-1124. In one embodiment, the culture is harvested and measured for expression of the transgene of interest using flow cytometry, enzyme-linked immunosorbent assay (ELISA), ELISPOT, or other assay known in the art.

In another embodiment, ELISA is used to measure antibody isotype production, e.g., IgM, or a product of the transgene of interest. In certain embodiments, IgG determinations are made using commercially available antibodies, such as goat anti-human IgG, as capture antibody followed by detection using any of a variety of appropriate detection reagents such as biotinylated goat antihuman Ig, streptavidin alkaline phosphatase and substrate.

In certain embodiments, the differentiated B cells are cultured under conditions and for sufficient time periods such that the number of cells is 1, 10, 25, 50, 75, 100, 125, 150, 175, 200, 250, 300, 350, 400, 450, 500, 550, 600, 650, 700, 750, 800, 850, 900, 950, 1000 fold or more greater than the number of B cells at the start of culture. In one embodiment, the number of cells is 10-1000 fold greater, including consecutive integers therein, than the number of differentiated B cells at the start of culture. For example, an expanded differentiated B cell population is at least 10 fold greater than the initial isolated differentiated B cell population. In another embodiment, the expanded differentiated B cell population is at least 100 fold greater than the initial isolated B cell population. In one embodiment, the expanded B cell population is at least 500 fold greater than the initial isolated differentiated B cell population.

### Transfection of B Cells

In one embodiment, the differentiated B cells are transfected with a transgene. Exemplary methods for transfecting differentiated B cells are provided in WO 2014/152832 and WO 2016/100932, both of which are incorporated herein by reference in their entireties. Transfection of differentiated B cells may be accomplished using any of a variety of methods available in the art to introduce DNA or RNA into a differentiated B cell. Suitable techniques may include calcium phosphate transfection, DEAE-Dextran, electroporation, pressure-mediated transfection or “cell squeezing” (e.g., CellSqueeze microfluidic system, SQZ Biotechnologies), liposome-mediated transfection and transduction using retrovirus or other virus, e.g., vaccinia. See, e.g., Graham et al., 1973, Virology 52:456; Sambrook et al., 2001, Molecular Cloning, a Laboratory Manual, Cold Spring Harbor Laboratories; Davis et al., 1986, Basic Methods in Molecular Biology, Elsevier; Chu et al., 1981, Gene 13:197; U.S. Pat. No. 5,124,259; U.S. Pat. No. 5,297,983; U.S. Pat. No. 5,283,185; U.S. Pat. No. 5,661,018; U.S. Pat. No. 6,878,548; U.S. Pat. No. 7,799,555; U.S. Pat. No. 8,551,780; and U.S. Pat. No. 8,633,029. One example of a commercially available electroporation technique suitable for B cells is the Nucleofector™ transfection technology.

Transfection may take place prior to or during in vitro culture of the isolated B cells in the presence of one or more activating and/or differentiating factors described above. For example, cells are transfected on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 of in vitro culture. In one embodiment, cells are transfected on day 1, 2, or 3 of in vitro culture. In a particular embodiment, cells are transfected on day 2. For example, cells are electroporated on day 2 of in vitro culture for delivery of, e.g., a plasmid, a transposon, a minicircle, or a self-replicating RNA. In another embodiment, cells are transfected on day 4, 5, 6, or 7 of in vitro culture. In a particular embodiment, cells are transfected on day 6 of in vitro culture. In another embodiment, cells are transfected on day 5 of in vitro culture.

In one embodiment, cells are transfected prior to activation. In another embodiment, cells are transfected during activation. In one embodiment, cells are transfected after activation. In one embodiment, cells are transfected prior to differentiation. In another embodiment, cells are transfected during differentiation. In one embodiment, cells are transfected after differentiation.

In one embodiment, a non-viral vector is used to deliver DNA or RNA to memory B cells and/or plasma cells. Examples of non-viral vectors include, without limitation, transposons (e.g., Sleeping Beauty transposon system), zinc-finger nucleases (ZFNs), transcription activator-like effector nucleases (TALENs), clustered regularly interspaced short palindromic repeats (CRISPRs), minicircles, replicons, artificial chromosomes (e.g., bacterial artificial chromosomes, mammalian artificial chromosomes, and yeast artificial chromosomes), plasmids, cosmids, and bacteriophage.

In some embodiments, the integration of the transgene utilizes one or more of a zinc finger nuclease, a transcription activator-like effector nuclease (TALEN), and/or a CRISPR/Cas system including, but not limited to, the CRISPR/Cas9 system. In some embodiments, the target integration comprises a CRISPR PRIME editing system.

In one embodiment, a method of transfecting a differentiated B cell comprises electroporating the differentiated B cell prior to contacting the differentiated B cell with a vector. In one embodiment, cells are electroporated on day 1, 2, 3, 4, 5, 6, 7, 8, or 9 of in vitro culture. In one embodiment, cells are electroporated on day 2 of in vitro culture for delivery of a plasmid. In one embodiment, cells are transfected using a transposon on day 1, 2, 3, 4, 5, 6, 7, 8, or 9 of in vitro culture. In another embodiment, cells are transfected using a minicircle on day 1, 2, 3, 4, 5, 6, 7, 8, or 9 of in vitro culture. In one embodiment, electroporation of a Sleeping Beauty transposon takes place on day 2 of in vitro culture.

In one embodiment, the differentiated B cells are contacted with a vector comprising a nucleic acid of interest operably linked to a promoter, under conditions sufficient to transfect at least a portion of the differentiated B cells. In one embodiment the differentiated B cells are contacted with a vector comprising a nucleic acid of interest operably linked to a promoter, under conditions sufficient to transfect at least 5% of the differentiated B cells. In a further embodiment, the differentiated B cells are contacted with a vector under conditions sufficient to transfect at least 5%, 10%, 15%, 20%, 25%, 30%, 35%, 40%, 45%, 50%, 55%, 60%, 70%, 75%, 80%, 85%, 90%, 95%, 96%, 97%, 98%, 99% or even 100% of the B cells. In one particular embodiment, the differentiated B cells, cultured in vitro as described herein, are transfected, in which case the cultured differentiated B cells are contacted with a vector as described herein under conditions sufficient to transfect at least 5%, 10% 15%, 20%, 25%, 30%, 35%, 40%, 45%, 50%, 55%, 60%, 70%, 75%, 80%, 85%, 90%, 95%, 96%, 97%, 98%, 99% or even 100% of the B cells.

Viral vectors may be employed to transduce memory B cells and/or plasma cells. Examples of viral vectors include, without limitation, adenovirus-based vectors, adeno-associated virus (AAV)-based vectors, retroviral vectors, retroviral-adenoviral vectors, and vectors derived from herpes simplex viruses (HSVs), including amplicon vectors, replication-defective HSV and attenuated HSV (see, e.g., Krisky, Gene Ther. 5:1517-30, 1998; Pfeifer, Annu. Rev. Genomics Hum. Genet. 2:177-211, 2001, each of which is incorporated by reference in its entirety).

In one embodiment, cells are transduced with a viral vector (e.g., a lentiviral vector) on day 1, 2, 3, 4, 5, 6, 7, 8, or 9 of in vitro culture. In a particular embodiment, cells are transduced with a viral vector on day 5 of in vitro culture. In one embodiment, the viral vector is a lentivirus. In one embodiment, cells are transduced with a measles virus pseudotyped lentivirus on day 1 of in vitro culture.

In one embodiment, differentiated B cells are transduced with retroviral vectors using any of a variety of known techniques in the art (see, e.g., Science 12 Apr. 1996 272:263-267; Blood 2007, 99:2342-2350; Blood 2009, 1 13:1422-1431; Blood 2009 Oct. 8; 1 14 (15): 3173-80; Blood. 2003;101 (6): 2167-2174; Current Protocols in Molecular Biology or Current Protocols in Immunology, John Wiley & Sons, New York, N.Y. (2009)). Additional description of viral transduction of B cells may be found in WO 2011/085247 and WO 2014/152832, each of which is herein incorporated by reference in its entirety.

For example, PBMCs, B-or T-lymphocytes from donors, and other B cell cancer cells such as B-CLLs may be isolated and cultured in IMDM medium or RPMI 1640 (GibcoBRL Invitrogen, Auckland, New Zealand) or other suitable medium as described herein, either serum-free or supplemented with serum (e.g., 5-10% FCS, human AB serum, and serum substitutes) and penicillin/streptomycin and/or other suitable supplements such as transferrin and/or insulin. In one embodiment, cells are seeded at 1×10⁵ cells in 48-well plates and concentrated vector added at various doses that may be routinely optimized by the skilled person using routine methodologies. In one embodiment, B cells are transferred to an MS5 cell monolayer in RPMI supplemented with 10% AB serum, 5% FCS, 50 ng/ml rhSCF, 10 ng/ml rhlL-15 and 5 ng/ml rhlL-2 and medium refreshed periodically as needed. As would be recognized by the skilled person, other suitable media and supplements may be used as desired.

Certain embodiments relate to the use of retroviral vectors, or vectors derived from retroviruses. “Retroviruses” are enveloped RNA viruses that are capable of infecting animal cells, and that utilize the enzyme reverse transcriptase in the early stages of infection to generate a DNA copy from their RNA genome, which is then typically integrated into the host genome. Examples of retroviral vectors Moloney murine leukemia virus (MLV)-derived vectors, retroviral vectors based on a Murine Stem Cell Virus, which provides long-term stable expression in target cells such as hematopoietic precursor cells and their differentiated progeny (see, e.g., Hawley et al., PNAS USA 93:10297-10302, 1996; Keller et al., Blood 92:877-887, 1998), hybrid vectors (see, e.g., Choi, et al., Stem Cells 19:236-246, 2001), and complex retrovirus-derived vectors, such as lentiviral vectors.

In one embodiment, the differentiated B cells are contacted with a retroviral vector comprising a nucleic acid of interest operably linked to a promoter, under conditions sufficient to transduce at least a portion of the differentiated B cells. In one embodiment the differentiated B cells are contacted with a retroviral vector comprising a nucleic acid of interest operably linked to a promoter, under conditions sufficient to transduce at least 2% of the differentiated B cells. In a further embodiment, the differentiated B cells are contacted with a vector under conditions sufficient to transduce at least 2%, 3%, 4%, 5%, 10%, 15%, 20%, 25%, 30%, 35%, 40%, 45%, 50%, 55%, 60%, 70%, 75%, 80%, 85%, 90%, 95%, 96%, 97%, 98%, 99% or even 100% of the resting B cells. In one particular embodiment, the differentiated and activated B cells, cultured in vitro as described herein, are transduced, in which case the cultured differentiated/activated B cells are contacted with a vector as described herein under conditions sufficient to transduce at least 2%, 3%, 4%, 5%, 10% 15%, 20%, 25%, 30%, 35%, 40%, 45%, 50%, 55%, 60%, 70%, 75%, 80%, 85%, 90%, 95%, 96%, 97%, 98%, 99% or even 100% of the differentiated and activated B cells.

In certain embodiments, prior to transduction, the cells are prestimulated with Staphylococcus Aureus Cowan (SAC; Calbiochem, San Diego, CA) and/or IL-2 at appropriate concentrations known to the skilled person and routinely optimized. Other B cell activating factors (e.g., PMA), as are known to the skilled artisan and described herein may be used.

As noted above, certain embodiments employ lentiviral vectors. The term “lentivirus” refers to a genus of complex retroviruses that are capable of infecting both dividing and non-dividing cells. Examples of lentiviruses include HIV (human immunodeficiency virus; including HIV type 1, and HIV type 2), visna-maedi, the caprine arthritis-encephalitis virus, equine infectious anemia virus, feline immunodeficiency virus (FIV), bovine immune deficiency virus (BIV), and simian immunodeficiency virus (SIV). Lentiviral vectors can be derived from any one or more of these lentiviruses (see, e.g., Evans et al., Hum Gene Ther. 10:1479-1489, 1999; Case et al., PNAS USA 96:2988-2993, 1999; Uchida et al., PNAS USA 95:1 1939-1 1944, 1998; Miyoshi et al., Science 283:682-686, 1999; Sutton et al., J Virol 72:5781-5788, 1998; and Frecha et al., Blood. 1 12:4843-52, 2008, each of which is incorporated by reference in its entirety).

It has been documented that resting T and B cells can be transduced by a VSVG-coated LV carrying most of the HIV accessory proteins (vif, vpr, vpu, and nef) (see e.g., Frecha et al., 2010 Mol. Therapy 18:1748). In certain embodiments the retroviral vector comprises certain minimal sequences from a lentivirus genome, such as the HIV genome or the SIV genome. The genome of a lentivirus is typically organized into a 5′ long terminal repeat (LTR) region, the gag gene, the pol gene, the env gene, the accessory genes (e.g., nef, vif, vpr, vpu, tat, rev) and a 3′ LTR region. The viral LTR is divided into three regions referred to as U3, R (repeat) and U5. The U3 region contains the enhancer and promoter elements, the U5 region contains the polyadenylation signals, and the R region separates the U3 and U5 regions. The transcribed sequences of the R region appear at both the 5′ and 3′ ends of the viral RNA (see, e.g., “RNA Viruses: A Practical Approach” (Alan J. Cann, Ed., Oxford University Press, 2000); O Narayan, J. Gen. Virology. 70:1617-1639, 1989; Fields et al., Fundamental Virology Raven Press., 1990; Miyoshi et al., J Virol. 72:8150-7,1998; and U.S. Pat. No. 6,013,516, each of which is incorporated by reference in its entirety). Lentiviral vectors may comprise any one or more of these elements of the lentiviral genome, to regulate the activity of the vector as desired, or, they may contain deletions, insertions, substitutions, or mutations in one or more of these elements, such as to reduce the pathological effects of lentiviral replication, or to limit the lentiviral vector to a single round of infection.

Typically, a minimal retroviral vector comprises certain 5′LTR and 3′LTR sequences, one or more genes of interest (to be expressed in the target cell), one or more promoters, and a cis-acting sequence for packaging of the RNA. Other regulatory sequences can be included, as described herein and known in the art. The viral vector is typically cloned into a plasmid that may be transfected into a packaging cell line, such as a eukaryotic cell (e.g., 293-HEK), and also typically comprises sequences useful for replication of the plasmid in bacteria.

In certain embodiments, the viral vector comprises sequences from the 5′ and/or the 3′ LTRs of a retrovirus such as a lentivirus. The LTR sequences may be LTR sequences from any lentivirus from any species. For example, they may be LTR sequences from HIV, SIV, FIV or BIV. Preferably the LTR sequences are HIV LTR sequences.

In certain embodiments, the viral vector comprises the R and U5 sequences from the 5′ LTR of a lentivirus and an inactivated or “self-inactivating” 3′ LTR from a lentivirus. A “self-inactivating 3′ LTR” is a 3′ long terminal repeat (LTR) that contains a mutation, substitution or deletion that prevents the LTR sequences from driving expression of a downstream gene. A copy of the U3 region from the 3′ LTR acts as a template for the generation of both LTR's in the integrated provirus. Thus, when the 3′ LTR with an inactivating deletion or mutation integrates as the 5′ LTR of the provirus, no transcription from the 5′ LTR is possible. This eliminates competition between the viral enhancer/promoter and any internal enhancer/promoter. Self-inactivating 3′ LTRs are described, for example, in Zufferey et al., J Virol. 72:9873-9880, 1998; Miyoshi et al., J Virol. 72:8150-8157, 1998; and Iwakuma et al., Wro/ogy 261:120-132, 1999, each of which is incorporated by reference in its entirety. Self-inactivating 3′ LTRs may be generated by any method known in the art. In certain embodiments, the U3 element of the 3′ LTR contains a deletion of its enhancer sequence, preferably the TATA box, Spl and/or NF-kappa B sites. As a result of the self-inactivating 3′ LTR, the provirus that is integrated into the host cell genome will comprise an inactivated 5′ LTR.

The vectors provided herein typically comprise a gene that encodes a protein (or other molecule, such as siRNA) that is desirably expressed in one or more target cells. In a viral vector, the gene of interest is preferably located between the 5′ LTR and 3′ LTR sequences. Further, the gene of interest is preferably in a functional relationship with other genetic elements, for example, transcription regulatory sequences such as promoters and/or enhancers, to regulate expression of the gene of interest in a particular manner once the gene is incorporated into the target cell. In certain embodiments, the useful transcriptional regulatory sequences are those that are highly regulated with respect to activity, both temporally and spatially.

In certain embodiments, one or more additional genes may be incorporated as a safety measure, mainly to allow for the selective killing of transfected target cells within a heterogeneous population, such as within a human patient. In one exemplary embodiment, the selected gene is a thymidine kinase gene (TK), the expression of which renders a target cell susceptible to the action of the drug gancyclovir. In a further embodiment, the suicide gene is a caspase 9 suicide gene activated by a dimerizing drug (see, e.g., Tey et al., Biology of Blood and Marrow Transplantation 13:913-924, 2007).

In certain embodiments, a gene encoding a marker protein may be placed before or after the primary gene in a viral or non-viral vector to allow for identification and/or selection of cells that are expressing the desired protein. Certain embodiments incorporate a fluorescent marker protein, such as green fluorescent protein (GFP) or red fluorescent protein (RFP), along with the primary gene of interest. If one or more additional reporter genes are included, IRES sequences or 2A elements may also be included, separating the primary gene of interest from a reporter gene and/or any other gene of interest.

Certain embodiments may employ genes that encode one or more selectable markers. Examples include selectable markers that are effective in a eukaryotic cell or a prokaryotic cell, such as a gene for a drug resistance that encodes a factor necessary for the survival or growth of transformed host cells grown in a selective culture medium. Exemplary selection genes encode proteins that confer resistance to antibiotics or other toxins, e.g., G418, hygromycin B, puromycin, zeocin, ouabain, blasticidin, ampicillin, neomycin, methotrexate, or tetracycline, complement auxotrophic deficiencies, or supply may be present on a separate plasmid and introduced by co-transfection with the viral vector. In one embodiment, the gene encodes for a mutant dihydrofolate reductase (DHFR) that confers methotrexate resistance. Certain other embodiments may employ genes that encode one or cell surface receptors that can be used for tagging and detection or purification of transfected cells (e.g., low-affinity nerve growth factor receptor (LNGFR) or other such receptors useful as transduction tag systems. See e.g., Lauer et al., Cancer Gene Ther. 2000 March;7 (3): 430-7.

Certain viral vectors such as retroviral vectors employ one or more heterologous promoters, enhancers, or both. In certain embodiments, the U3 sequence from a retroviral or lentiviral 5′ LTR may be replaced with a promoter or enhancer sequence in the viral construct. Certain embodiments employ an “internal” promoter/enhancer that is located between the 5′ LTR and 3′ LTR sequences of the viral vector, and is operably linked to the gene of interest.

A “functional relationship” and “operably linked” mean, without limitation, that the gene is in the correct location and orientation with respect to the promoter and/or enhancer, such that expression of the gene will be affected when the promoter and/or enhancer is contacted with the appropriate regulatory molecules. Any enhancer/promoter combination may be used that either regulates (e.g., increases, decreases) expression of the viral RNA genome in the packaging cell line, regulates expression of the selected gene of interest in an infected target cell, or both.

A promoter is an expression control element formed by a DNA sequence that permits polymerase binding and transcription to occur. Promoters are untranslated sequences that are located upstream (5′) of the start codon of a selected gene of interest (typically within about 100 to 1000 bp) and control the transcription and translation of the coding polynucleotide sequence to which they are operably linked. Promoters may be inducible or constitutive. Inducible promoters initiate increased levels of transcription from DNA under their control in response to some change in culture conditions, such as a change in temperature. Promoters may be unidirectional or bidirectional. Bidirectional promoters can be used to co-express two genes, e.g., a gene of interest and a selection marker. Alternatively, a bidirectional promoter configuration comprising two promoters, each controlling expression of a different gene, in opposite orientation in the same vector may be utilized.

A variety of promoters are known in the art, as are methods for operably linking the promoter to the polynucleotide coding sequence. Both native promoter sequences and many heterologous promoters may be used to direct expression of the selected gene of interest. Certain embodiments employ heterologous promoters, because they generally permit greater transcription and higher yields of the desired protein as compared to the native promoter.

Certain embodiments may employ heterologous viral promoters. Examples of such promoters include those obtained from the genomes of viruses such as polyoma virus, fowlpox virus, adenovirus, bovine papilloma virus, avian sarcoma virus, cytomegalovirus, a retrovirus, hepatitis-B virus and Simian Virus 40 (SV40). Certain embodiments may employ heterologous mammalian promoter, such as the actin promoter, an immunoglobulin promoter, a heat-shock promoter, or a promoter that is associated with the native sequence of the gene of interest. Typically, the promoter is compatible with the target cell, such as an activated B-lymphocyte, a plasma B cell, a memory B cell or other lymphocyte target cell.

Certain embodiments may employ one or more of the RNA polymerase II and III promoters. A suitable selection of RNA polymerase III promoters can be found, for example, in Paule and White. Nucleic Acids Research., Vol. 28, U.S. Plant Pat. No. 1,283-1298, 2000, which is incorporated by reference in its entirety. RNA polymerase II and III promoters also include any synthetic or engineered DNA fragments that can direct RNA polymerase II or III, respectively, to transcribe its downstream RNA coding sequences. Further, the RNA polymerase II or III (Pol II or III) promoter or promoters used as part of the viral vector can be inducible. Any suitable inducible Pol II or III promoter can be used with the methods described herein. Exemplary Pol II or III promoters include the tetracycline responsive promoters provided in Ohkawa and Taira, Human Gene Therapy, Vol. 11, pp 577-585, 2000; and Meissner et al., Nucleic Acids Research, Vol. 29, U.S. Plant Pat. No. 1,672-1682, 2001, each of which is incorporated by reference in its entirety.

Non-limiting examples of constitutive promoters that may be used include the promoter for ubiquitin, the CMV promoter (see, e.g., Karasuyama et al., J. Exp. Med. 169:13, 1989), the beta-actin (see, e.g., Gunning et al., PNAS USA 84:4831-4835, 1987), the elongation factor-1 alpha (EF-1 alpha) promoter, the CAG promoter, and the pgk promoter (see, e.g., Adra et al., Gene 60:65-74, 1987); Singer-Sam et al., Gene 32:409-417, 1984; and Dobson et al., Nucleic Acids Res. 10:2635-2637, 1982, each of which is incorporated by reference). Non-limiting examples of tissue specific promoters include the lck promoter (see, e.g., Garvin et al., Mol. Cell Biol. 8:3058-3064, 1988; and Takadera et al., Mol. Cell Biol. 9:2173-2180, 1989), the myogenin promoter (Yee et al., Genes and Development 7:1277-1289. 1993), and the thyl promoter (see, e.g., Gundersen et al., Gene 1 13:207-214, 1992).

Additional examples of promoters include the ubiquitin-C promoter, the human mu heavy chain promoter or the Ig heavy chain promoter (e.g., MH), and the human kappa light chain promoter or the Ig light chain promoter (e.g., EEK), which are functional in B-lymphocytes. The MH promoter contains the human mu heavy chain promoter preceded by the iEmu enhancer flanked by matrix association regions, and the EEK promoter contains the kappa light chain promoter preceded an intronic enhancer (iEkappa), a matrix associated region, and a 3′ enhancer (3Ekappa) (see, e.g., Luo et al., Blood. 1 13:1422-1431, 2009, and U.S. Patent Application Publication No. 2010/0203630). Accordingly, certain embodiments may employ one or more of these promoter or enhancer elements. In some embodiments, the EEK promoter is a sequence that is 95% similar to the sequence disclosed in positions 343-1245 of SEQ ID NO: 26. In some embodiments, the EEK promoter sequence is disclosed in 343-1245 of SEQ ID NO: 26.

As noted above, certain embodiments employ enhancer elements, such as an internal enhancer, to increase expression of the gene of interest. Enhancers are cis-acting elements of DNA, usually about 10 to 300 bp in length, that act on a promoter to increase its transcription. Enhancer sequences may be derived from mammalian genes (e.g., globin, elastase, albumin, «-fetoprotein, insulin), such as the teu enhancer, the tek intronic enhancer, and the 3′ EK enhancer. Also included are enhancers from a eukaryotic virus, including the SV40 enhancer on the late side of the replication origin (bp 100-270), the cytomegalovirus early promoter enhancer, the polyoma enhancer on the late side of the replication origin, and adenovirus enhancers. Enhancers may be spliced into the vector at a position 5′ or 3′ to the antigen-specific polynucleotide sequence, but are preferably located at a site 5′ from the promoter. Persons of skill in the art will select the appropriate enhancer based on the desired expression pattern.

In certain embodiments, promoters are selected to allow for inducible expression of the gene. A number of systems for inducible expression are known in the art, including the tetracycline responsive system and the lac operator-repressor system. It is also contemplated that a combination of promoters may be used to obtain the desired expression of the gene of interest. The skilled artisan will be able to select a promoter based on the desired expression pattern of the gene in the organism and/or the target cell of interest.

Certain viral vectors contain cis-acting packaging sequences to promote incorporation of the genomic viral RNA into the viral particle. Examples include psi-sequences. Such cis-acting sequences are known in the art. In certain embodiments, the viral vectors described herein may express two or more genes, which may be accomplished, for example, by incorporating an internal promoter that is operably linked to each separate gene beyond the first gene, by incorporating an element that facilitates co-expression such as an internal ribosomal entry sequence (IRES) element (U.S. Pat. No. 4,937,190, incorporated by reference) or a 2A element, or both. Merely by way of illustration, IRES or 2A elements may be used when a single vector comprises sequences encoding each chain of an immunoglobulin molecule with a desired specificity. For instance, the first coding region (encoding either the heavy or light chain) may be located immediately downstream from the promoter, and the second coding region (encoding the other chain) may be located downstream from the first coding region, with an IRES or 2A element located between the first and second coding regions, preferably immediately preceding the second coding region. In other embodiments, an IRES or 2A element is used to co-express an unrelated gene, such as a reporter gene, a selectable marker, or a gene that enhances immune function. Examples of IRES sequences that can be used include, without limitation, the IRES elements of encephalomyelitis virus (EMCV), foot-and-mouth disease virus (FMDV), Theiler's murine encephalomyelitis virus (TMEV), human rhinovirus (HRV), coxsackievirus (CSV), poliovirus (POLIO), Hepatitis A virus (HAV), Hepatitis C virus (HCV), and Pestiviruses (e.g., hog cholera virus (HOCV) and bovine viral diarrhea virus (BVDV)) (see, e.g., Le et al., Virus Genes 12:135-147, 1996; and Le et al., Nuc. Acids Res. 25:362-369, 1997, each of which is incorporated by reference in their entirety). One example of a 2A element includes the F2A sequence from foot-and-mouth disease virus. In some embodiments, the 2a element is a T2A sequence. In some embodiments, the T2A is disclosed in SEQ ID NO: 21.

In certain embodiments, the vectors provided herein also contain additional genetic elements to achieve a desired result. For example, certain viral vectors may include a signal that facilitates nuclear entry of the viral genome in the target cell, such as an HIV-1 flap signal. As a further example, certain viral vectors may include elements that facilitate the characterization of the provirus integration site in the target cell, such as a tRNA amber suppressor sequence. Certain viral vectors may contain one or more genetic elements designed to enhance expression of the gene of interest. For example, a woodchuck hepatitis virus responsive element (WRE) may be placed into the construct (see, e.g., Zufferey et al., J. Virol. 74:3668-3681, 1999; and Deglon et al., Hum. Gene Ther. 11:179-190, 2000, each of which is incorporated by reference in its entirety). As another example, a chicken beta-globin insulator may also be included in the construct. This element has been shown to reduce the chance of silencing the integrated DNA in the target cell due to methylation and heterochromatinization effects. In addition, the insulator may shield the internal enhancer, promoter and exogenous gene from positive or negative positional effects from surrounding DNA at the integration site on the chromosome. Certain embodiments employ each of these genetic elements. In another embodiment, the viral vectors provided herein may also contain a Ubiquitous Chromatin Opening Element (UCOE) to increase expression (see e.g., Zhang F, et al., Molecular Therapy: The journal of the American Society of Gene Therapy 2010 September;18 (9): 1640-9.)

In certain embodiments, the viral vectors (e.g., retroviral, lentiviral) provided herein are “pseudo-typed” with one or more selected viral glycoproteins or envelope proteins, mainly to target selected cell types. Pseudo-typing refers to generally to the incorporation of one or more heterologous viral glycoproteins onto the cell-surface virus particle, often allowing the virus particle to infect a selected cell that differs from its normal target cells. A “heterologous” element is derived from a virus other than the virus from which the RNA genome of the viral vector is derived. Typically, the glycoprotein-coding regions of the viral vector have been genetically altered such as by deletion to prevent expression of its own glycoprotein. Merely by way of illustration, the envelope glycoproteins gp41 and/or gp120 from an HIV-derived lentiviral vector are typically deleted prior to pseudo-typing with a heterologous viral glycoprotein.

In certain embodiments, the viral vector is pseudo-typed with a heterologous viral glycoprotein that targets B lymphocytes. In certain embodiments, the viral glycoprotein allows selective infection or transduction of resting or quiescent B lymphocytes. In certain embodiments, the viral glycoprotein allows selective infection of B lymphocyte plasma cells, plasmablasts, and activated B cells. In certain embodiments, the viral glycoprotein allows infection or transduction of quiescent B lymphocytes, plasmablasts, plasma cells, and activated B cells. In certain embodiments, viral glycoprotein allows infection of B cell chronic lymphocyte leukemia cells. In one embodiment, the viral vector is pseudo-typed with VSV-G. In another embodiment, the heterologous viral glycoprotein is derived from the glycoprotein of the measles virus, such as the Edmonton measles virus. Certain embodiments pseudo-type the measles virus glycoproteins hemagglutinin (H), fusion protein (F), or both (see, e.g., Frecha et al., Blood. 1 12:4843-52, 2008; and Frecha et al., Blood. 1 14:3173-80, 2009, each of which is incorporated by reference in its entirety). In one embodiment, the viral vector is pseudo-typed with gibbon ape leukemia virus (GALV). In further embodiments, the viral vector comprises an embedded antibody binding domain, such as one or more variable regions (e.g., heavy and light chain variable regions) which serves to target the vector to a particular cell type.

Generation of viral vectors can be accomplished using any suitable genetic engineering techniques known in the art, including, without limitation, the standard techniques of restriction endonuclease digestion, ligation, transformation, plasmid purification, PCR amplification, and DNA sequencing, for example as described in Sambrook et al. (Molecular Cloning: A Laboratory Manual. Cold Spring Harbor Laboratory Press, N.Y. (1989)), Coffin et al. (Retroviruses. Cold Spring Harbor Laboratory Press, N.Y. (1997)) and “RNA Viruses: A Practical Approach” (Alan J. Cann, Ed., Oxford University Press, (2000)).

Any variety of methods known in the art may be used to produce suitable retroviral particles whose genome comprises an RNA copy of the viral vector. As one method, the viral vector may be introduced into a packaging cell line that packages the viral genomic RNA based on the viral vector into viral particles with a desired target cell specificity. The packaging cell line typically provides in trans the viral proteins that are required for packaging the viral genomic RNA into viral particles and infecting the target cell, including the structural gag proteins, the enzymatic pol proteins, and the envelope glycoproteins.

In certain embodiments, the packaging cell line stably expresses certain necessary or desired viral proteins (e.g., gag, pol) (see, e.g., U.S. Pat. No. 6,218,181, herein incorporated by reference). In certain embodiments, the packaging cell line is transiently transfected with plasmids that encode certain of the necessary or desired viral proteins (e.g., gag, pol, glycoprotein), including the measles virus glycoprotein sequences described herein. In one exemplary embodiment, the packaging cell line stably expresses the gag and pol sequences, and the cell line is then transfected with a plasmid encoding the viral vector and a plasmid encoding the glycoprotein. Following introduction of the desired plasmids, viral particles are collected and processed accordingly, such as by ultracentrifugation to achieve a concentrated stock of viral particles. Exemplary packaging cell lines include 293 (ATCC CCL X), HeLa (ATCC CCL 2), D17 (ATCC CCL 183), MDCK (ATCC CCL 34), BHK (ATCC CCL-10) and Cf2Th (ATCC CRL 1430) cell lines.

### DEFINITIONS

In this disclosure, “comprises,” “comprising,” “containing,” “having,” and the like can have the meaning ascribed to them in U.S. Patent law and can mean “includes,” “including,” and the like; the terms “consisting essentially of” or “consists essentially” likewise have the meaning ascribed in U.S. Patent law and these terms are open-ended, allowing for the presence of more than that which is recited so long as basic or novel characteristics of that which is recited are not changed by the presence of more than that which is recited, but excludes prior art embodiments.

Unless specifically stated or obvious from context, as used herein, the terms “a,” “an,” and “the” are understood to be singular or plural.

Unless specifically stated or obvious from context, as used herein, the term “or” is understood to be inclusive.

As used herein, the term “about,” unless indicated otherwise, refers to the recited value, e.g., amount, dose, temperature, time, percentage, etc., +10%, +9%, +8%, +7%, +6%, +5%, +4%, +3%, +2%, or +1%.

As used herein, the term “subject” herein to refer to any mammal, including humans, domestic and farm animals, and zoo, sports, and pet animals, such as dogs, horses, cats, and agricultural use animals including cattle, sheep, pigs, and goats. One preferred mammal is a human, including adults, children, and the elderly. A subject may also be a pet animal, including dogs, cats and horses. Preferred agricultural animals would be pigs, cattle and goats. A “patient” is a human subject.

The phrases “therapeutically effective amount” and “effective amount” and the like, as used herein, indicate an amount necessary to administer to a patient, or to a cell, tissue, or organ of a patient, to achieve a therapeutic effect, such as an ameliorating or alternatively a curative effect. The effective amount is sufficient to elicit the biological or medical response of a cell, tissue, system, animal, or human that is being sought by a researcher, veterinarian, medical doctor, or clinician. Determination of the appropriate effective amount or therapeutically effective amount is within the routine level of skill in the art.

The terms “administering,” “administer,” “administration” and the like, as used herein, refer to any mode of transferring, delivering, introducing, or transporting a therapeutic agent to a subject in need of treatment with such an agent. Such modes include, but are not limited to, intraocular, oral, topical, intravenous, intraperitoneal, intramuscular, intradermal, intranasal, and subcutaneous administration.

As used herein “gene of interest,” “gene,” or “nucleic acid of interest” refers to a transgene to be expressed in the target transfected cell. While the term “gene” may be used, this is not to imply that this is a gene as found in genomic DNA and is used interchangeably with the term “nucleic acid”. Generally, the nucleic acid of interest provides suitable nucleic acid for encoding a therapeutic agent and may comprise cDNA or DNA and may or may not include introns, but generally does not include introns. As noted elsewhere, the nucleic acid of interest is operably linked to expression control sequences to effectively express the protein of interest in the target cell. In certain embodiments, the vectors described herein may comprise one or more genes of interest, and may include 2, 3, 4, or 5 or more genes of interest, such as for example, the heavy and light chains of an immunoglobulin that may be organized using an internal promoter as described herein.

The recitation “polynucleotide” or “nucleic acid” as used herein designates mRNA, RNA, CRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA and RNA. The nucleic acid or gene of interest may be any nucleic acid encoding a protein of interest.

As used herein, unless as otherwise described with regard to viral vectors, “vector” means a nucleic acid molecule capable of transporting another nucleic acid to which it has been linked.

Exemplary vectors include plasmids, minicircles, transposons (e.g., Sleeping Beauty transposon), yeast artificial chromosomes, self-replicating RNAs, and viral genomes. Certain vectors can autonomously replicate in a host cell, while other vectors can be integrated into the genome of a host cell and thereby are replicated with the host genome. In addition, certain vectors are referred to herein as “recombinant expression vectors” (or simply, “expression vectors”), which contain nucleic acid sequences that are operatively linked to an expression control sequence and, therefore, are capable of directing the expression of those sequences. In certain embodiments, expression constructs are derived from plasmid vectors. Illustrative constructs include modified pNASS vector (Clontech, Palo Alto, CA), which has nucleic acid sequences encoding an ampicillin resistance gene, a polyadenylation signal and a T7 promoter site; pDEF38 and pNEF38 (CMC ICOS Biologies, Inc.), which have a CHEF1 promoter; and pD18 (Lonza), which has a CMV promoter. Other suitable mammalian expression vectors are well known (see, e.g., Ausubel et al., 1995; Sambrook et al., supra; see also, e.g., catalogs from Invitrogen, San Diego, CA; Novagen, Madison, WI; Pharmacia, Piscataway, NJ).

The recitation “migration” as used herein means the capacity to traffic, localize, or target to a particular location or tissue. Certain cells as described herein have a “migratory capacity” or ability to traffic, localize, or target to a particular location or tissue.

### INCORPORATION BY REFERNCE

All references, articles, publications, patents, patent publications, and patent applications cited herein are incorporated by reference in their entireties for all purposes. However, mention of any reference, article, publication, patent, patent publication, and patent application herein is not, and should not be, taken as acknowledgment or any form of suggestion that they constitute valid prior art or form part of the common general knowledge in any country in the world.

### EXAMPLES

The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees

Celsius, and pressure is at or near atmospheric. Standard abbreviations can be used, e.g., bp, base pair(s); kb, kilobase(s); pl, picoliter(s); s or sec, second(s); min, minute(s); h or hr, hour(s); aa, amino acid(s); nt, nucleotide(s); and the like.

The practice of the present invention will employ, unless otherwise indicated, conventional methods of protein chemistry, biochemistry, recombinant DNA techniques and pharmacology, within the skill of the art

### Example 1: Modified B Cell Migration to Bone Tissue

In order to determine if modified B cells can be used to deliver a therapeutic agent in vivo to a variety of tissues, MPS I mice were given a series of 3 doses of IDUA producing differentiated B cells. MPS I mice were given a series of 3 doses of 1×10⁷ differentiated B cells engineered to produce IDUA (or no cells as a control) in the presence of CD4+ memory T cells (or no cells as a control) on day 0 and IDUA enzyme activity levels measured in serum through day 56. Specifically, MPS I mice were infused i.p. with 3×10⁶ CD4+ T cells at day −7 and then 10⁷ pKT2/EEK-IDUA transposed differentiated B cells (approximately 10% IDUA+ by intracellular staining) either i.p. or i.v. on day 0. The animals were given additional infusions of 107 pKT2/EEK-IDUA transposed B cells by the same route of administration on days 21 and 42 after the first injection.

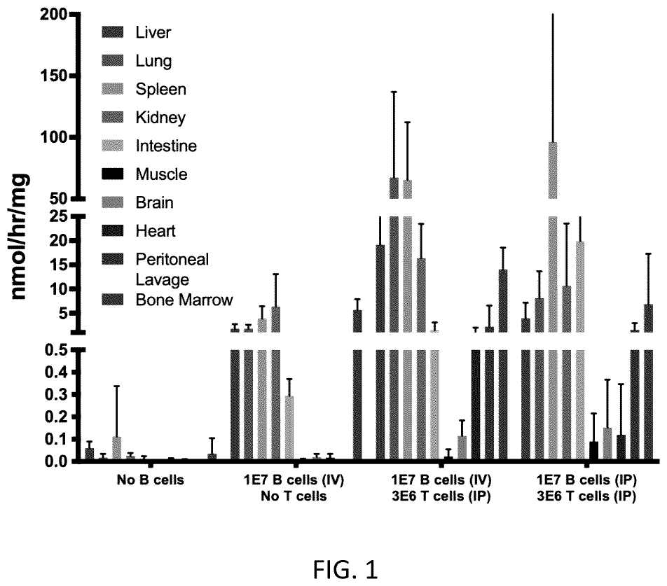

MPS I mice were given a dosage regiment comprising 3 doses of 1×10⁷ differentiated B cells engineered to produce IDUA (or no cells as a control) in the presence of CD4+ T cells (or no cells as a control). Animals were euthanized and tissues harvested at 56 days post the first differentiated B cell infusion, and IDUA enzyme activity levels measured in the liver, lung, spleen, kidney, intestine, muscle, brain, heart, peritoneal lavage, and bone marrow (FIG. 1).

Flow cytometry showed 20% to 35% human CD 45+ and 2% to 10% CD19+ cells in spleen and lymph nodes of animals infused with human T and IDUA expressing B cells. There was substantial metabolic cross-correction observed by the IDUA activities restored in peripheral tissues, including bone.

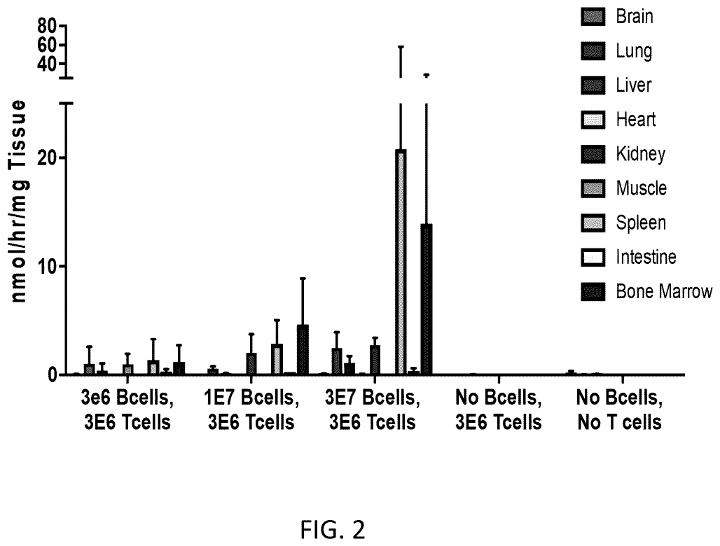

Next, MPS I mice were given a dosage regiment comprising 1 dose of 1×10⁷ differentiated B cells engineered to produce IDUA (or no cells as a control) in the presence of CD4+ T cells (or no cells as a control. Animals were euthanized and tissues harvested at 65 days post the first differentiated B cell infusion, and IDUA enzyme activity levels measured in the liver, lung, spleen, kidney, intestine, muscle, brain, heart, peritoneal lavage, and bone marrow (FIG. 2). Mice given a dosage of 1 dose of 1×107 differentiated B cells demonstrated little bone marrow IDUA tissue activity.

In addition, MPS I mice were given a dosage regiment comprising 1 dose of 3×10⁷ differentiated B cells engineered to produce IDUA (or no cells as a control) in the presence of CD4+ T cells (or no cells as a control. Animals were euthanized and tissues harvested at 85 days post the first differentiated B cell infusion, and IDUA enzyme activity levels measured in the liver, lung, spleen, kidney, intestine, muscle, brain, heart, peritoneal lavage, and bone marrow (FIG. 2). Mice given a dosage of 1 dose of 3×10⁷ differentiated B cells demonstrated bone marrow IDUA tissue activity. Mice given a dosage of 1 dose of 1×10⁷ differentiated B cells demonstrated metabolic cross-correction observed by the IDUA activities restored in bone marrow (FIG. 2). Further examples are described in WO2018201071.

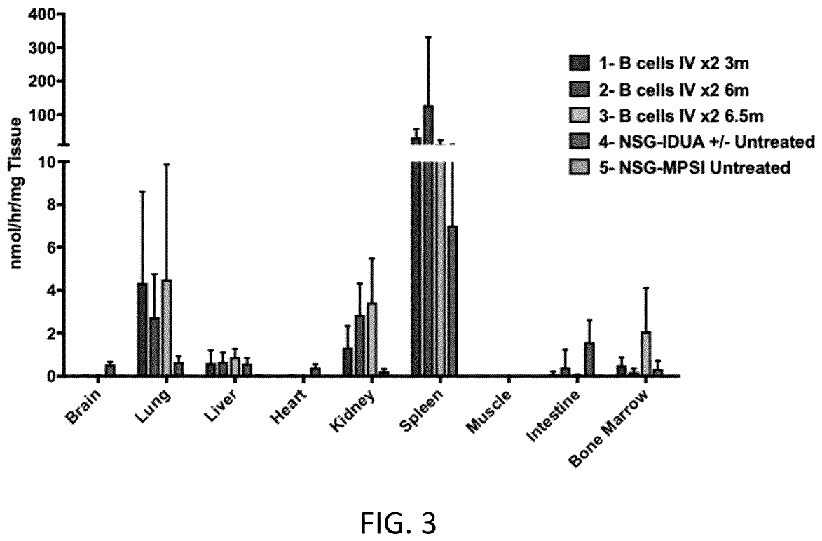

Tissue IDUA activity was determined after sacrifice at 3, 6, and 6.5 months (3 m, 6 m, and 6.5 m) after the second of two injections of 2×10⁷ B cells given 36 days apart (FIG. 3). Control groups (4 and 5) were split between the three harvest time points. All B cell treated mice received CD4+ T cells IP 7 days prior to the first B cell injection. Extracts were prepared from the tissues indicated on the horizontal axis and assayed for IDUA activity as described in WO2018201071. The mean +/− S.D. is shown for N=5 for the 3 and 6 month timepoints, N=3 for the 6.5 month timepoint. N=1,1, and 2 for IDUA+/− untreated controls at 3, 6 and 6.5 months. N=1, 1, and 0for MPSI untreated controls at 3, 6 and 6.5 months, respectively. Bone tissue IDUA activity is confirmed after 6.5 months after the second of the two injections.

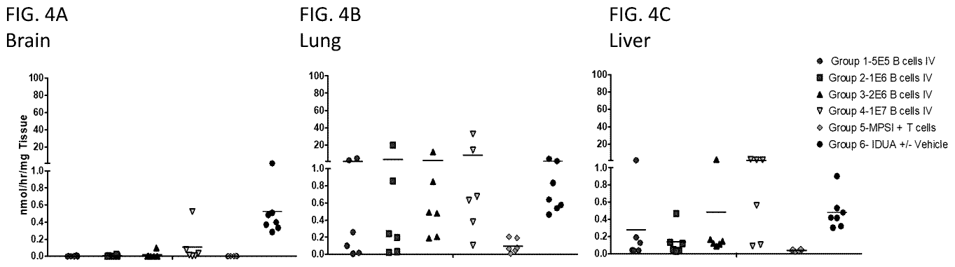

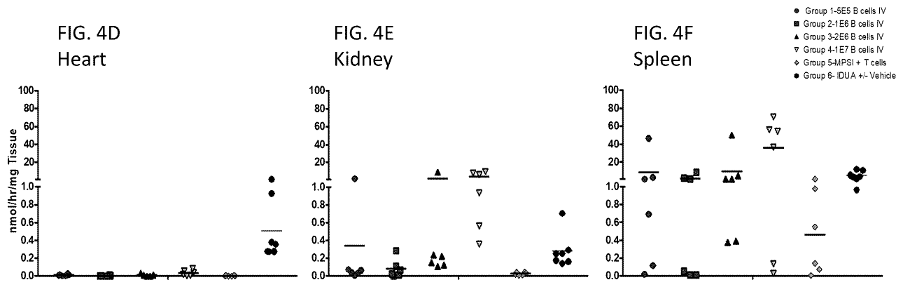

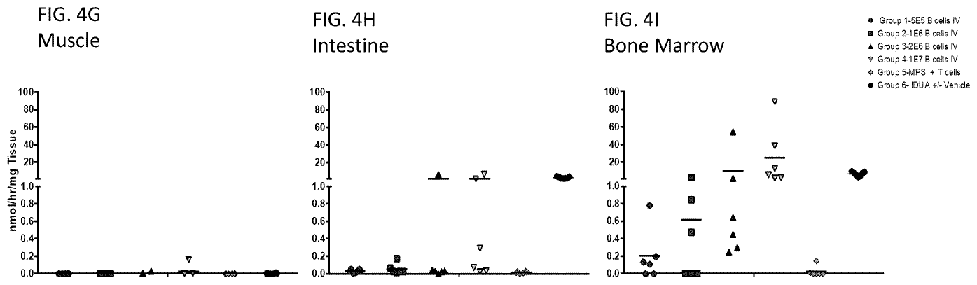

Tissue IDUA activity was determined after sacrifice at 2 months after B cell injection. There were six treatment groups: (1) 5E5 differentiated B cells, (2) 1E6 differentiated B cells, (3) 2E6 differentiated B cells, (4) 1E7 differentiated B cells, (5) MPSI untreated mice that only got T cells, and (6) vehicle treated mice (IDUA +/−). (FIG. 4). IDUA activity for brain (FIG. 4A), lung (FIG. 4B), liver (FIG. 4C), heart (FIG. 4D), kidney (FIG. 4E), spleen (FIG. 4F), muscle (FIG. 4G), intestine (FIG. 4H), and bone marrow (FIG. 41) was confirmed after 2 months after the injection of the range of B cell concentrations, in particular group 3 and 4.

### Example 2: Production of Exogenous HIV-Neutralizing Antibody and Antibody Fragment Expressing B Cells from Lenti-Virus (LV)

### Transduction and Activation of B Cells

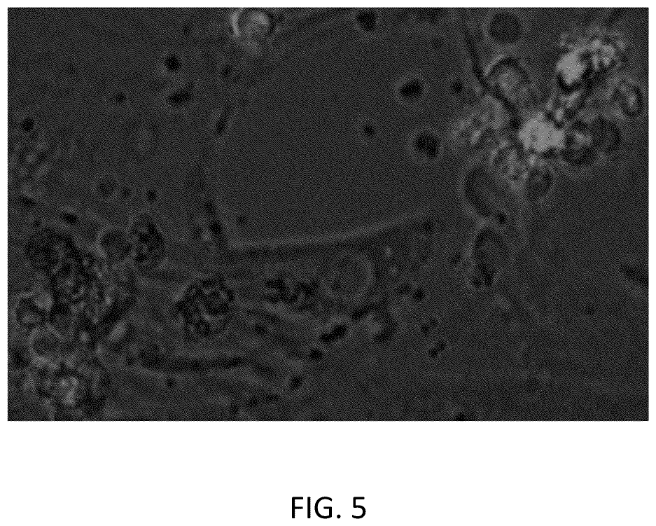

To demonstrate proof of concept of a B cell expressing an exogenous antibody, a measles virus-pseudotyped lenti-viurs (LV) encoding GFP under the control of the EEK promoter was utilized. B cells were isolated from human peripheral blood using a Ficoll gradient followed by depletion-based magnetic bead purification using the EasySep system (Stem Cell Technologies). The cells were pre-stimulated for 3 days in the Baltimore culture system, which involves plating of cells onto adherent stromal cells expressing low levels of CD40 ligand (feeder cells) in the presence of IL-2 (R&D), IL-10 (eBioscience), and CpG (Integrated Technologies). Specifically, 5×10⁴ stromal cells and 3×10⁴ B cells were plated in 1 mL of media in a 24-well plate (Corning). The cells were subsequently transduced with the modified LV vector carrying the gene for green fluorescent protein (GFP) using a multiplicity of infection (MOI) of 10. Following transduction, the cells were returned to the culture system and the cytokines refreshed every 2 days. FIG. 5 shows expression of GFP by activated B cells after 12 days in culture. Importantly, it should be noted that the EEK promoter is only active in plasmacytes after several days in culture and not in resting B cells. Thus, the GFP-positive cells represent plasma cells. These cells demonstrate a much larger morphology than the resting cells from which they were derived. The background of FIG. 5 shows the adherent feeder cells.

### Production of the HIV-Neutralizing Antibody B12

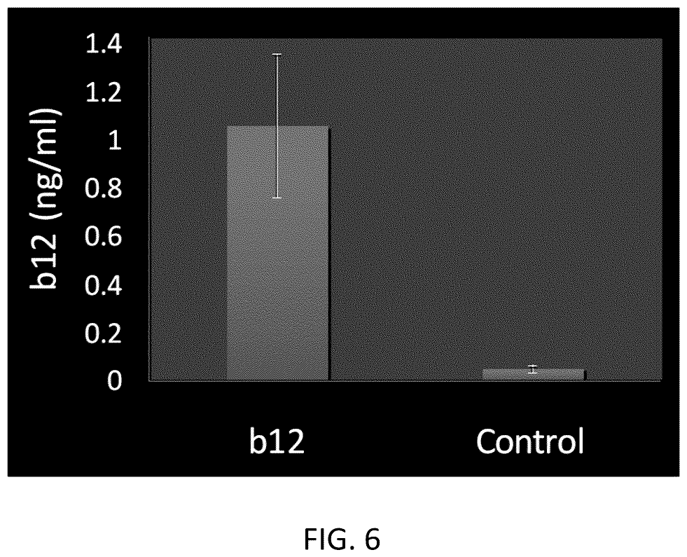

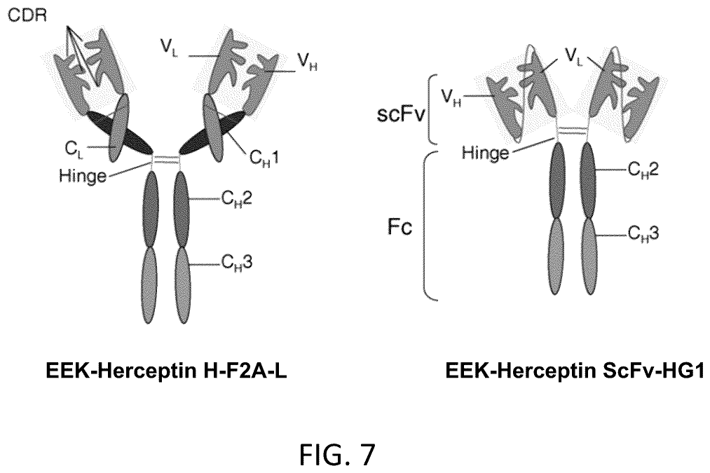

Cells were isolated, transduced, and cultured as described above, this time using a LV vector encoding the b12 antibody. Using a Luminex detection device, a concentration of approximately 1 ng/ml of the b12 antibody was observed in the culture media from the transduced cells (FIG. 6). Specifically, beads coated with GP140 were used to measure b12 and concentration determined using a standard curve based on recombinant b12. These studies demonstrate the ability to generate specific antibody-producing B cells from whole blood.

### Example 3: Production of Exogenous Anti-Cancer Antibody and Antibody Fragment Expressing B Cells

Sleeping Beauty transposon and transposase constructs for transposition and expression of anti-HER-2 antibody (SEQ ID NO: 9) and anti-HER2 scFv-G1Fc (SEQ ID NO: 1) were generated. Transposons were assembled to achieve therapeutic protein gene integration and expression in differentiated B cells. Transposon were designed to integrate into the native heavy chain locus. The transposon is comprised of the EEK promoter and EF1 a promoter to achieve high level expression in B cells (SEQ ID NO: 26 is an example of the anti-HER-2 antibody transposon sequence). To enrich for therapeutic protein expressing cells, the transposon further comprises a bifunctional bicistron comprising the therapeutic antibody and a selection marker (EGFRt or DHFR). The therapeutic protein comprises an epitope tag such as a HIS-tag, or MYC-TAG, or HA-tag, which allows for differentiation of the therapeutic protein from other human monoclonal antibodies produced in culture. The epitope tag allows for separation and/or purification of the therapeutic protein, e.g., anti-HER-2 antibody, from other human monoclonal antibodies produced in culture. In some embodiments, the therapeutic protein is isolated using standard chromatography and gel filtration methods.

To test for therapeutic protein transposition and expression, human B cells were isolated from human donors and expanded in culture, followed by electroporation of the transposon. Cell lysates were prepared post-electroporation and the presence of therapeutic protein was confirmed.

### Example 4: In Vitro Determination of Modified B Cell Antibody-Dependent Cell-Mediated Cytotoxicity And Complement-Dependent Cytotoxicity

Vectors (e.g., plasmid) comprising polynucleotide that encode for a monoclonal therapeutic antibody or a therapeutic scFv-G1Fc are transiently transfected in serum-free 293F cells. Cells are cultured and exogenous therapeutic protein is expressed. In some embodiments, the therapeutic protein is secreted or is membrane bound. The exogenously expressed monoclonal antibody or the exogenously expressed scFv-G1Fc are isolated and purified using a HIS-tag, or a MYC-tag, and/or a HA-tag. The tag allows for easy differentiation of exogenously expressed protein from other native human monoclonal antibodies. In some embodiments, the therapeutic protein is isolated using standard chromatography and gel filtration methods.

To confirm antibody-dependent cell-mediated cytotoxicity (ADCC) and complement-dependent cytotoxicity (CDC), a PSMA negative (PSMA-) LNCaP cell line and a PSMA positive (PSMA+) CHO cell line (target cells due to PSMA expression) are cultured.

ADCC and CDC are confirmed using a commercial reporter bioassay (for example, Promega ADCC Reporter Bioassay). ADCC and CDC activity is confirmed and quantified in a dose dependent manner for both the therapeutic antibody and the therapeutic scFv-G1Fc. In addition, no ADCC and CDC activity is confirmed for both the therapeutic antibody and the therapeutic scFv-G1Fc in the PSMA-cell line control.

To confirm the anti-PSMA effector function of the modified B cells which express a therapeutic antibody or a therapeutic scFv-G1Fc, an in vitro ADCC and CDC assay is carried out with PSMA+ target cells co-incubated with modified B cells that express the therapeutic antibody and the therapeutic scFv-G1Fc. ADCC and CDC are confirmed using a commercial reporter bioassay (for example, Promega ADCC Reporter Bioassay). ADCC and CDC activity is confirmed and quantified in a dose dependent (across titrations of B cells) manner of the modified B cells which express the therapeutic protein and the therapeutic scFv-G1Fc. In addition, no ADCC and CDC activity is confirmed for both the therapeutic antibody and the therapeutic scFv-G1Fc in the PSMA-cell line control.

### Example 5: In Vitro Determination of Antibody Binding and Effector Function

Direct binding of the therapeutic antibody and/or the therapeutic scFv-G1Fc in a dose response manner is confirmed. Direct binding of the therapeutic antibody and/or the therapeutic scFv-G1Fc is confirmed by flow cytometry and by surface plasmon resonance (SPR) methods. The binding constants for both the therapeutic antibody and/or the therapeutic scFv-G1Fc to PSMA is confirmed via SPR. SPR sensograms demonstrate high affinity of the produced therapeutic antibody and/or the therapeutic scFv-G1Fc.

### Example 6: In Vivo Determination of Therapeutic Efficacy of a B Cell Expressing Anti-Psma in A Xenograft Tumor Cells Mouse Model

A population of modified B cells (as prepared following Example 4) which express either an exogenous therapeutic antibody or an exogenous therapeutic scFv-G1Fc are injected into PSMA+ xenograft mouse models comprising human tumor cells to confirm therapeutic efficacy. Xenograft mice are generated through either orthotopic or heterotopic implantation of human tumor cell lines. In some embodiments, LNCaP or CHO cells are implanted in mice.

Mice received an intravenous (i.v.) bolus injection or a subcutaneous injection or an intraperitoneal injection of a variable (titration) amount of a population of modified B cells. Whole blood samples were collected over a time series and antibody concentrations are determined using standard assay means (e.g., ELISA). Pharmacokinetic parameters are determined from the mean plasma concentrations.

In vivo studies are conducted to evaluate the antitumor activity of the modified B cell composition, injected into mice as a population of modified B cells. As a control, mice are also administered vehicle alone. Xenograft mice comprising variable amounts of implanted tumor cell lines are administered variable amounts of a population of the modified B cells. Treatment efficacy in terms of tumor formation and growth is determined by standard methods across the treatment groups.

A significant and dose dependent delay of tumor formation/growth is observed for mice treated with the modified B cell composition, as compared to the vehicle control.

### Example 7 In Vivo Determination of Therapeutic Efficacy of a B Cell Expressing Anti-Psma in a Patient-Derived Xenograft Mouse Model

Following the same procedure as Example 6, patient-derived human prostate cancer xenograft mice (i.e., PDX mice) are generated, and the therapeutic efficacy of the modified B cell composition is tested. Cancer metastasis to mouse bone is confirmed prior to treatment.

In vivo studies are conducted to evaluate the antitumor activity of the modified B cell composition, injected into mice as a population of modified B cells. As a control, mice are also administered vehicle alone. Human prostate cancer PDX mice comprising variable amounts of implanted tumor cell lines are administered variable amounts of a population of the modified B cells. Treatment efficacy in terms of tumor formation and growth is determined by standard methods across the treatment groups.

A significant and dose dependent delay of tumor formation/growth in bone is observed for mice treated with the modified B cell composition, as compared to the vehicle control.

### Example 8: HER-2 Binding Demonstrated with Human ISP Cells (CD38+, CD138+, CD20−) Engineered with Sleeping Beauty Constructs Encoding Herceptin Monoclonal Antibody, Herceptin SCFV-, or J591-SCFV

Modified human differentiated B cells (CD38+, CD138+, and CD20−) (i.e., human ISP cells, or hISPs) were generated by the transduction of a Sleeping Beauty (SB) construct encoding a form of Herceptin (anti-HER-2) (SEQ ID NO: 26) or anti-PSMA (J591) scFv (SEQ ID NO:27). The SB constructs encoded a monoclonal Herceptin full-length heavy/light antibody (SEQ ID NO: 1), a Herceptin scFv (SEQ ID NO: 9), or a anti-PSMA (J591) scFv (SEQ ID NO: 25), respectively. All protein expression was driven by the EEK promoter. In addition, CHO cells were transduced with either HER2 or PSMA.

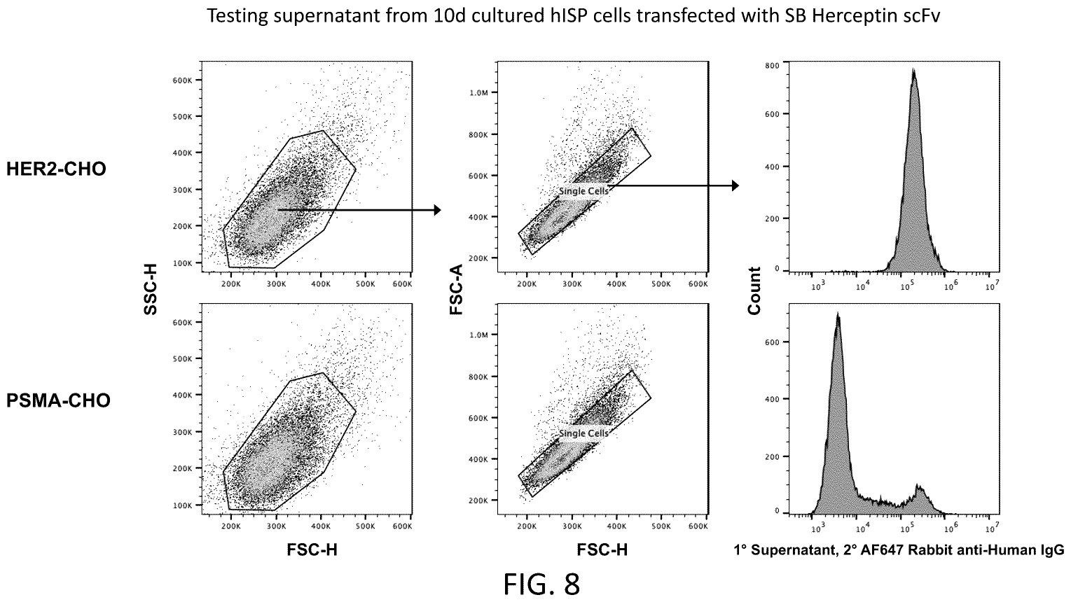

After SB construct transduction into human differentiated B cells (hISPs), supernatant protein expression after 10-day culture (post-transduction with SB construct), protein expression was analyzed via flow cytometry. As seen in FIG. 8, the supernatant of the Herceptin scFv expressing differentiated B cells (hISP) cells showed significant cell-surface binding to stably HER2-expressing CHO cells, but not PSMA-expressing CHO cells.

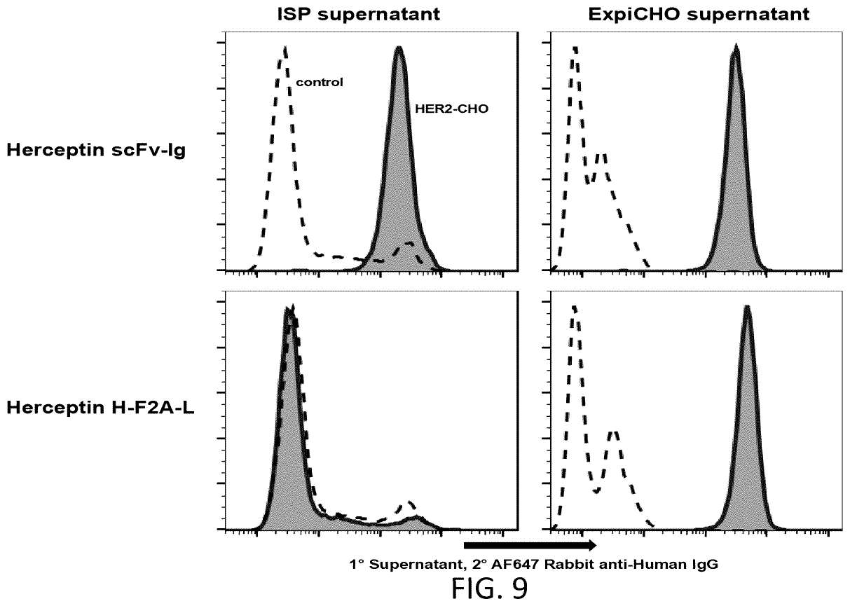

FIG. 9 shows binding results, as measured by flow cytometry, of cell culture supernatant from modified differentiated B cells expressing the Herceptin scFv-(SEQ ID NO: 1) and the supernatant from modified B cells expressing full-length Herceptin antibody (SEQ ID NO: 9) or transiently transfected SF CHO cells with the same. In particular, the results demonstrate full-length Herceptin antibody and Herceptin scFv, respectively, produced within the ExpiCHO cell line demonstrated HER-2 binding as measured by flow cytometry. The supernatant from the Herceptin scFv-Ig from either the ISP or the ExpiCHO cells exhibited binding to HER2-CHO cells when compared to control.

Surprisingly, the full-length Herceptin monoclonal antibody was only able to be expressed by the ExpiCHO cells, and it was not expressed by the differentiated B cells (hISP). This is seen by the bottom right panel in FIG. 9 (ExpiCHO supernatant) as compared to the bottom left panel (hISP or differentiated B cell) of FIG. 9. In the right panel, there is strong binding showing that the full antibody can be synthesized and formed correctly when transiently transfected into the CHO cells, whereas the left panel shows that there is no binding from the ISP supernatant showing that there was some issue in the formulation of synthetization of the full length antibody. On the other hand, the scFv of the same antibody showed similar binding when transfected into the ExpiCHO cells (top right panel of FIG. 9) and into the differentiated B cells (top left panel of FIG. 9).

Not to be limited by any theory, but the lack of detectable binding of HER-2 by full-length Herceptin antibody produced from the differentiated B cells (hISPs) could possibly be due to post-translational mispairing with the endogenous immunoglobulin light chains in the B cell.

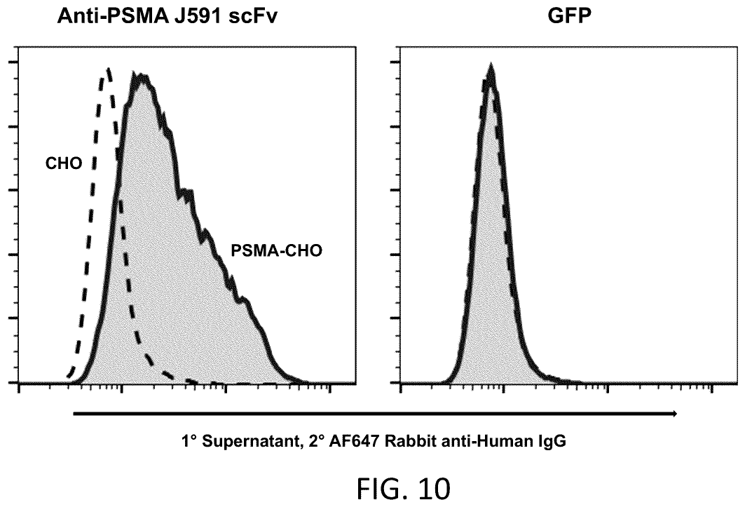

Lastly, based on the results with production of the Herceptin scFv, the production of the J591-scFv was tested. As seen in FIG. 10, cell culture supernatants from SB modified differentiated B cells, expressing either the anti-PSMA J591 scFv (left) or GFP (right) were subjected to flow cytometry analysis. The supernatant from the J591 scFv expressing cells exhibited binding to PSMA-CHO cells when compared to control CHO, whereas that from the GFP engineered cells did not.

### EMBODIMENTS OF THE INVENTION

Embodiment 1: A modified B cell that expresses at least one therapeutic protein; wherein the modified B cell is CD38+, CD138+, and CD20−; and is capable of homing to bone marrow.

Embodiment 2: The modified B cell of embodiment 1, wherein the B cell is CD78+, IL-6R+, and CD27++.

Embodiment 3: The modified B cell of embodiment 2, wherein the B cell is CD138^{high} (CD138++).

Embodiment 4: The modified B cell of any one of the preceding embodiments, wherein the at least one therapeutic protein is capable of binding a tumor associated antigen.

Embodiment 5: The modified B cell of any one of the preceding embodiments, wherein the therapeutic protein is an exogenous antigen-specific antibody or an exogenous antigen-binding fragment thereof.

Embodiment 6: The modified B cell of embodiment 5, wherein the exogenous antigen-specific antibody or an exogenous antigen-binding fragment thereof has a modified fragment crystallizable (Fc) region.

Embodiment 7: The modified B cell of any one of the preceding embodiments, wherein the therapeutic protein is an anti-PSMA antibody or an anti-PSMA antibody fragment thereof, or the therapeutic protein is an anti-HER-2 antibody or an anti-HER-2 antibody fragment thereof, or the therapeutic protein is an anti-MUC1 antibody or an anti-MUC1 antibody fragment thereof, or the therapeutic protein is an anti-NYESO-1 antibody or an anti-NYESO-1 antibody fragment thereof, or the therapeutic protein is an anti-CEA antibody or an anti-CEA antibody fragment thereof, or the therapeutic protein is an anti-MAGE-Al antibody or an anti-MAGE-A1 antibody fragment thereof, or the therapeutic protein is an anti-α-fetoprotein antibody or an anti-α-fetoprotein antibody fragment thereof, or the therapeutic protein is an anti-CA 19-9 antibody or an anti-CA 19-9 antibody fragment thereof.

Embodiment 8: The modified B cell of embodiment 7, wherein the anti-PSMA antibody is 3/A12 or J591.

Embodiment 9: The modified B cell of embodiment 7, wherein the anti-PSMA antibody fragment is a scFv or scFv fusion.

Embodiment 10: The modified B cell of embodiment 9, wherein the scFv is A5 scFv or a derivative of A5 scFv or wherein the scFv is J591 scFv or a derivative of J591 scFv.

Embodiment 11: The modified B cell of embodiment 7, wherein the anti-HER-2 antibody is any one of trastuzumab (Herceptin), pertuzumab, pertuzumab-tratuzumab-hyaluronidase-zzxf, or margetuximab.

Embodiment 12: The modified B cell of embodiment of 7, wherein the anti-HER-2 antibody fragment is a scFv or scFv fusion.

Embodiment 13: The modified B cell of any one of the preceding embodiments, wherein the anti-PSMA or anti-HER-2 therapeutic protein is expressed from a transgene.

Embodiment 14: The modified B cell of embodiment 13, wherein the transgene is incorporated into the B cell genome.

Embodiment 15: The modified B cell of embodiment 14, wherein the transgene is incorporated in the immunoglobulin heavy chain locus.

Embodiment 16: The modified B cell of any one of the preceding embodiments, wherein the modified B cell secretes one or more of a cytokine, a signaling molecule, or a small molecule.

Embodiment 17: A method of producing a population of modified B cells, the method comprising:
(a) isolating pan-B cells, memory B cells, switch memory B cells, plasmablasts, or plasma cells from a sample, thereby obtaining an isolated B cell population;
(b) culturing the isolated B cell population in vitro with one or more B cell activating factors, thereby obtaining an expanded B cell population;
(c) transfecting or transducing the expanded B cell population with a transgene; and
(d) differentiating the expanded B cell population in vitro with one or more B cell activating factors, thereby obtaining a modified B cell composition.

Embodiment 18: The method of embodiment 17, wherein the transfecting or transducing step further comprises enriching the expanded B cell population using a selectable marker.

Embodiment 19: The method of embodiment 18, wherein the selectable marker is selected from the group consisting of a fluorescent marker protein, a drug resistance factor, and a surface marker.

Embodiment 20: The method of any one of embodiments 17-19, wherein the transfecting step or transducing step comprises electroporation, lipofection, non-viral transduction, or viral transduction.

Embodiment 21: The method of embodiment 20, wherein the non-viral transduction comprises a non-viral vector.

Embodiment 22: The method of embodiment 21, wherein the non-viral vector is a transposon.

Embodiment 23: The method of embodiment 22, wherein the transposon is a sleeping beauty transposon.

Embodiment 24: The method of any one of the preceding embodiments, wherein the transgene encodes an anti-PSMA or anti-HER-2 therapeutic protein.

Embodiment 25: The method of claim 24, wherein the anti-PSMA therapeutic protein or the anti-HER-2 therapeutic protein is an antigen-specific antibody or a scFv.

Embodiment 26: The method of any one of embodiments 17-25, wherein the transposon comprises an EEK or an EFla promoter.

Embodiment 27: The method of any one of embodiments 17-26, wherein the anti-PSMA therapeutic protein or anti-HER-2 therapeutic protein further comprises a HIS-tag, a MYC-tag, or a hemagglutinin tag.

Embodiment 28: The method of any one of embodiments 17-27, wherein the transgene is incorporated into the genome.

Embodiment 29: The method of embodiment 28, wherein the transgene is incorporated into the genome at the immunoglobulin heavy chain locus.

Embodiment 30: The method of embodiment 29, wherein the transgene is incorporated into the genome using a nuclease, recombinase, transposase, or integrase.

Embodiment 31: The method of embodiment 30, wherein the nuclease is a Cas nuclease, a meganuclease, a zinc-finger nuclease, or a transcription activator like effector nuclease.

Embodiment 32: A method of treating a subject having a cancer in bone marrow

comprising administering to the subject a therapeutically effective amount of the population of modified B cells of any of embodiments 1-16.

Embodiment 33: The method of embodiment 32, wherein the cancer is metastatic cancer, wherein the metastatic cancer is prostate cancer, breast cancer, lung cancer, brain cancer, kidney cancer, skin cancer, multiple myeloma, thyroid cancer, stomach cancer, lymphoma, leukemia, bone cancer, cervical cancer, ovarian cancer, bladder cancer, eye cancer, testicular cancer, pancreatic cancer, or sarcoma.

Embodiment 34: The method of embodiment 33, wherein the cancer is a tumor.

Embodiment 36: The method of any one of embodiments 32-34, wherein the tumor is a primary tumor.

1. A modified differentiated B cell that expresses an scFv, wherein
a) the modified differentiated B cell is CD38+, CD138+, and CD20−;
b) the modified differentiated B cell is CD78+, IL-6R+, and CD27++; and/or
c) the modified differentiated B cell is CD138^{high} (CD138++).

2-3. (canceled)

4. The modified differentiated B cell of claim 1, wherein the scFv is capable of binding a tumor associated antigen.

5. The modified differentiated B cell of claim 4, wherein the scFv is an anti-PSMA, an anti-HER-2, an anti-MUC1, an anti-NYESO-1, an anti-CEA, an anti-MAGE-A1, an anti-α-fetoprotein, or an anti-CA 19-9 scFv.

6. The modified differentiated B cell of claim 5, wherein
a) the scFv is A5 scFv or a derivative of A5 scFv
b) the scFv is J591 scFv or a derivative of J591 scFv; or
c) the scFv is derived from on any one of trastuzumab (Herceptin), pertuzumab, pertuzumab-tratuzumab-hyaluronidase-zzxf, or margetuximab.

7. (canceled)

8. The modified differentiated B cell of claim 5, wherein the anti-PSMA or anti-HER-2 scFv is expressed from a transgene, and wherein the transgene is incorporated into the modified differentiated B cell genome.

9. (canceled)

10. The modified differentiated B cell of claim 8, wherein the transgene is incorporated in a immunoglobulin heavy chain locus of the modified differentiated B cell.

11. The modified differentiated B cell of claim 1, wherein the modified differentiated B cell secretes one or more of a cytokine, a signaling molecule, or a small molecule.

12. The modified differentiated B cell of claim 11, wherein the modified differentiated B cell is capable of homing to bone marrow.

13. The modified differentiated B cell of claim 12, wherein the modified differentiated B cell engrafts within the bone marrow.

14. A method of producing a population of modified differentiated B cells, the method comprising:
(a) isolating pan-B cells, memory B cells, switch memory B cells, plasmablasts, or plasma cells from a sample, thereby obtaining an isolated B cell population;
(b) culturing the isolated B cell population in vitro with one or more B cell activating factors, thereby obtaining an expanded B cell population;
(c) transfecting or transducing the expanded B cell population with a gene encoding an scFv; and
(d) differentiating the expanded B cell population in vitro with one or more B cell activating factors, thereby obtaining a modified differentiated B cell composition.

15. The method of claim 14, wherein step (c) further comprises enriching the expanded B cell population using a selectable marker.

16. The method of claim 15, wherein the selectable marker is selected from the group consisting of a fluorescent marker protein, a drug resistance factor, and a surface marker.

17. The method of claim 14, wherein step (c) comprises electroporation, lipofection, non-viral transduction, or viral transduction.

18. The method of claim 17, wherein the non-viral transduction comprises a non-viral vector.

19. The method of claim 18, wherein the non-viral vector is a transposon.

20. The method of claim 19, wherein the transposon is a sleeping beauty transposon.

21. The method of claim 19, wherein the gene encoding an scFv encodes an anti-PSMA scFv or anti-HER-2 scFv, wherein the gene is incorporated into a genome of the modified differentiated B cell.

22. (canceled)

23. The method of claim 21, wherein the anti-PSMA scFv or anti-HER-2 scFv further comprises a HIS-tag, a MYC-tag, or a hemagglutinin tag, wherein the transposon comprises an EEK or EFla promoter.

24. (canceled)

25. The method of claim 21, wherein the gene encoding an scFv is incorporated into the genome at the immunoglobulin heavy chain locus of the modified differentiated B cell.

26. The method of claim 25, wherein the gene encoding an scFv is incorporated into the genome using a nuclease, recombinase, transposase, or integrase.

27. The method of claim 26, wherein the nuclease is a Cas nuclease, a meganuclease, a zinc-finger nuclease, or a transcription activator like effector nuclease.

28. A method of treating a subject having a cancer in bone marrow comprising administering to the subject a therapeutically effective amount of a population of the modified differentiated B cells of claim 1.

29. The method of claim 28, wherein the cancer is metastatic cancer, wherein the metastatic cancer is prostate cancer, breast cancer, lung cancer, brain cancer, kidney cancer, skin cancer, multiple myeloma, thyroid cancer, stomach cancer, lymphoma, leukemia, bone cancer, cervical cancer, ovarian cancer, bladder cancer, eye cancer, testicular cancer, pancreatic cancer, or sarcoma.

30. (canceled)

31. The method of claim 29, wherein the cancer is a primary tumor.
